(19) United States
(12) Patent Application Publication
(10) Pub. No.: US 2021/0010016 A1
(43) Pub. Date: Jan. 14, 2021

(54) PLANTS WITH ALTERED PRODUCTION OF BIOMASS CONSTITUENTS AND METHODS OF USE

(71) Applicant: The Board of Regents of the University of Oklahoma, Norman, OK (US)

(72) Inventors: Laura E. Bartley, Pullman, WA (US); Kangmei Zhao, Menlo Park, CA (US)

(21) Appl. No.: 16/946,822

(22) Filed: Jul. 8, 2020

Related U.S. Application Data
(60) Provisional application No. 62/872,990, filed on Jul. 11, 2019.

(51) Int. Cl. C12N 15/82; C07K 14/415

(52) U.S. Cl. CPC C12N 15/8255; C07K 14/415; C12N 15/8246

(57) ABSTRACT

Nucleic acid constructs, and cells, plants, and plant parts containing such constructs, for modifying content of biomass constituents (e.g, lignin, hemicellulose, and/or cellulose) in plants via altered expression of certain transcription factors (regulators). Lignin, hemicellulose, and/or cellulose content can be decreased by increasing expression of certain negative regulators, or by decreasing expression of positive regulators. Alternatively, lignin, hemicellulose, and/or cellulose content can be increased by decreasing expression of negative regulators, or by increasing expression of positive regulators.

### CROSS-REFERENCE TO RELATED APPLICATIONS

This application claims priority to U.S. Provisional Application No. 62/872,990, filed on Jul. 11, 2019, which is expressly incorporated herein by reference in its entirety.

### GOVERNMENT SUPPORT

This invention was made with government support under Grant Number DE-SC0006904 awarded by the Department of Energy Plant Feedstock Genomics Program. The government has certain rights in the invention.

### BACKGROUND

Lignin is a complex heterogeneous aromatic polymer which renders membranes impermeable and reinforces the walls of certain plants cells. Lignin is formed by polymerization of at least three different monolignols which are synthesized in a multistep pathway, each step in the pathway being catalyzed by a different enzyme. It has been shown that manipulation of the number of copies of genes encoding certain enzymes results in modification of the amount of lignin produced.

Beyond its role in the structure and development of plants, lignin represents a major component of the terrestrial biomass and assumes a major economic and ecological significance. Notably, lignin is a limiting factor of the digestibility and nutritional yield of fodder plants. For example, the digestibility of fodder plants by ruminants is inversely proportional to the content of lignin in these plants

Furthermore, lignin must be extracted from woody material for production of paper pulp in the paper industry. This extraction operation, which is necessary to obtain cellulose, is costly in energy and, secondly, causes pollution through the chemical compounds used for the extraction. Reducing the lignin content of the material used to make paper would represent an increase in yield and a substantial savings (chemical products) and would contribute to improving the environment (reduction in pollution).

In addition, lignin is a significant component of biomass which could be converted to fuel (such as ethanol) through the conversion of cellulosic biomass to ethanol. Lignin, hemicellulose, and cellulose fibers are intimately associated in the biomass of plants. Lignin can create a barrier that prevents cellulose degradation through either chemical methods or through the use of enzymes. The removal of lignin as well as hemicellulose is an important step in the process of converting cellulosic biomass to ethanol independent of the method of converting this biomass to fuel. Lignin poses a challenge to enzyme-based conversion of cellulosic biomass to fuel and one of the goals of biomass pretreatment is the removal of lignin. Many pre-treatments associated with the conversion of cellulosic material to ethanol remove the lignin component as well as other components from the plant biomass. Plants with reduced lignin content would be a more efficient biomass for the cellulosic conversion of plant biomass to fuel, such as ethanol.

Rice plant biomass is a promising lignocellulosic bioenergy crop due to its high biomass yield. However, a major barrier to its efficient conversion to fuel is the relatively low biodegradability of the lignin-enriched secondary cell wall which inhibits enzymatic degradation and thereby increases pretreatment costs, as noted above. Enhancing the biodegradability of rice plant biomass and other crops amenable to biofuel production by manipulation of lignin biosynthesis is one strategy for increasing biofuel availability.

### DETAILED DESCRIPTION

Improved biomass digestibility is an important trait both for more efficient utilization of feed by animals and for more efficient utilization of biomass for biorefining to fuels and other biomass-based products, such as plastics, and other carbon-based molecules. In at least certain embodiments, the present disclosure is therefore directed to nucleic acid constructs, and to cells and plants containing such constructs, for modifying content of biomass constituents (e.g, lignin, hemicellulose, and/or cellulose) in plants via altered expression of certain transcription factors (regulators) which have been newly discovered to affect lignin synthesis. Decreased lignin, hemicellulose, and/or cellulose content can be caused herein, for example, by increasing expression of negative regulators, or by decreasing expression of positive regulators, whereas increased lignin, hemicellulose, and/or cellulose content can be caused, for example, by decreasing expression of negative regulators, or by increasing expression of positive regulators.

In certain embodiments therefore, the present disclosure is directed to recombinant nucleic acid constructs, and plants, plant parts, and/or plant cells containing the constructs, and methods of their use, which encode transcription factors which negatively regulate production of lignin, hemicellulose, and/or cellulose. In certain embodiments of the constructs, the expression of such negative transcription factors is enhanced, while in certain other embodiments their expression is repressed.

In certain embodiments, the present disclosure is directed to recombinant nucleic acid constructs, and plants, plant parts, and/or plant cells containing the constructs, and methods of their use, which encode transcription factors which positively regulate production of lignin, hemicellulose, and/or cellulose. In certain embodiments of the constructs, the expression of such positive transcription factors is enhanced, while in certain other embodiments their expression is repressed.

Examples of plants, plant parts, or plant cells which may contain recombinant nucleic acid constructs of the present disclosure include, but are not limited to, switchgrass (Panicum virgatum), giant reed (Arundo donax), reed canarygrass (Phalaris arundinacea), MiscanthusXgiganteus, Miscanthus sp., Sericea lespedeza, corn, sugarcane, sorghum, millet, ryegrass, rye, timothy grass, Kochia (Kochia scoparia), soybean, alfalfa, clover, sunn hemp, kenaf, bahiagrass, bermudagrass, dallisgrass, pangolagrass, big bluestem, little bluestem, indiangrass, fescue, centipede grass (Eremochloa ophiuroides), Dactylis sp., Brachypodium distachyon, smooth bromegrass, orchardgrass, Kentucky bluegrass, poplar, rice, cotton, red sage, apple, Vitis vinifera, castor bean (Ricinus communis), hops (Humulus lupulus), Dahlia, orchid sp., mustards (e.g., Brassica rapa), kudzu (Pueraria lobata), wheat, eucalyptus, alder, and cedar.

Before describing various embodiments of the present disclosure in more detail by way of exemplary description, examples, and results, it is to be understood that the present disclosure is not limited in application to the details of methods, constructs, cells, and compositions as set forth in the following description. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that other embodiments of the inventive concepts may be practiced without these specific details. In other instances, features which are well known to persons of ordinary skill in the art have not been described in detail to avoid unnecessary complication of the description.

All patents, published patent applications, and non-patent publications referenced in any portion of this application, including U.S. Provisional Application No. 62/872,990, filed on Jul. 11, 2019, and all appendices filed therein, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the constructs, cells, compositions and methods used, or the variation that exists among the study objects. Further, in this detailed description and the appended claims, each numerical value (e.g., temperature or time) should be read once as modified by the term “about” (unless already expressly so modified), and then read again as not so modified unless otherwise indicated in context. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

Also, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1). Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 1-20, 10-50, 50-100, 100-1,000, 1,000-3,000, 2,000-4,000, etc.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The natural amino acids, where designated as such herein, include and may be referred to herein by the following designations: alanine: ala or A; arginine: arg or R; asparagine: asn or N; aspartic acid: asp or D; cysteine: cys or C; glutamic acid: glu or E; glutamine: gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

For purposes of classifying amino acids substitutions as conservative or nonconservative, in one non-limiting embodiment, amino acids are grouped in one embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of exemplary conservative amino acid substitutions have been constructed and are known in the art. In certain embodiments herein which reference possible substitutions, examples of interchangeable amino acids include, but are not limited to the following: arginine and lysine; glutamate and aspartate; serine and threonine; glutamine and asparagine; and valine, leucine and isoleucine. In other embodiments, the following substitutions can be made: Ala (A) by leu, ile, or val; Arg (R) by gln, asn, or lys; Asn (N) by his, asp, lys, arg, or gln; Asp (D) by asn, or glu; Cys (C) by ala, or ser; Gln (Q) by glu, or asn; Glu (E) by gln, or asp; Gly (G) by ala; His (H)by asn, gln, lys,or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe, or tyr; Tyr (Y) by trp, phe, thr, or ser; and Val (V) by ile, leu, met, phe, or ala.

Other considerations for amino acid substitutions include whether or not the residue is located in the interior of a protein or is solvent- (i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” a uracil “U” or a “C”). The term nucleobase also includes non-natural bases as described below. The term “nucleic acid” encompasses the terms “oligonucleotide” and “polynucleotide,” each as a subgenus of the term “nucleic acid.”

As used herein, the terms “complementary” or “complement” also refer to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

Non-limiting examples of DNA and protein sequences homologous to OsSND2 of the present disclosure are shown in Table A of U.S. Provisional Application No. 62/872,990, which table is included herein by reference its entirety. Non-limiting examples of DNA and protein sequences homologous to WACH1 of the present disclosure are shown in Table B of U.S. Provisional Application No. 62/872,990, which table is included herein by reference its entirety. Non-limiting examples of DNA and protein sequences homologous to OsMYB13a of the present disclosure are shown in Table C of U.S. Provisional Application No. 62/872,990, which table is included herein by reference its entirety. Non-limiting examples of DNA and protein sequences homologous to OsMYB13b of the present disclosure are shown in Table D of U.S. Provisional Application No. 62/872,990, which table is included herein by reference its entirety. Non-limiting examples of DNA and protein sequences homologous to WAP1 of the present disclosure are shown in Table E of U.S. Provisional Application No. 62/872,990, which table is included herein by reference its entirety. Non-limiting examples of DNA and protein sequences homologous to WAHL1 of the present disclosure are shown in Table F of U.S. Provisional Application No. 62/872.990, which table is included herein by reference its entirety. Non-limiting examples of DNA and protein sequences homologous to WAHD1 of the present disclosure are shown in Table G of U.S. Provisional Application No. 62/872,990, which table is included herein by reference its entirety.

The term “homologous” or “% identity” as used herein means a nucleic acid (or fragment thereof), or a protein (or a fragment thereof) having a degree of homology to the corresponding natural reference nucleic acid, or protein, that is at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% identical thereto. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). The default amino acid comparison matrix is blocks substitution matrix 62 (BLOSUM62) (Henikoff and Henikoff, PNAS 89(22) 10915-10919, (1992).

In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

Percentage sequence identities can be determined with protein sequences maximally aligned by the Kabat numbering convention. After alignment, if a particular polypeptide region is being compared with the same region of a reference polypeptide, the percentage sequence identity between the subject and reference polypeptide region is the number of positions occupied by the same amino acid in both the subject and reference polypeptide region divided by the total number of aligned positions of the two regions, with gaps not counted, multiplied by 100 to convert to percentage.

In one embodiment “% identity” represents the number of amino acids which are identical at corresponding positions in two sequences of a protein having the same or similar activity. For example, two amino acid sequences each having 100 residues will have at least 90% identity when 90 of the amino acids at corresponding positions are the same. Similarly, in one embodiment “% identity” represents the number of nucleotides which are identical at corresponding positions in two sequences of a nucleic acid encoding the same or similar polypeptides. For example, two nucleic acid sequences each having 100 nucleotides will have 90% identity when 90 of the nucleotides in homologous positions are the same.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Another algorithm is the WU-BLAST (Washington University BLAST) version 2.0 software (WU-BLAST version 2.0 executable programs for several UNIX platforms). This program is based on WU-BLAST version 1.4, which in tum is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3: 266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps.

As used herein, “hybridization,” “hybridizes” or “capable of hybridizing” is understood to mean the forming of a double or triple stranded molecule or a molecule with partial double or triple stranded nature. The term “anneal” as used herein is synonymous with “hybridize.” The term “hybridization,” “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Non-limiting applications include isolating a nucleic acid, such as a gene or a nucleic acid segment thereof, or detecting at least one specific mRNA transcript or a nucleic acid segment thereof, and the like. Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid, the length and nucleobase content of the target sequence, the charge composition of the nucleic acid, and to the presence or concentration of formamide, tetramethylammonium chloride or other solvent in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization to achieve varying degrees of selectivity of a nucleic acid towards a target sequence are used. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suit a particular application.

In certain embodiments herein, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

The term “encoding” as used herein refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the levelof a response. The response may be the expression of nucleic acid sequence or protein. The response may be compared with the level of a response in the absence of a treatment or compound, and/or compared with the level of a response in an otherwise identical but untreated entity, e.g., a cell or plant. The term encompasses perturbing and/or affecting a native signal or response thereby mediating a wild-type response in the entity.

A nucleic acid is “operably linked” when it is placed into a functional relationship with another nucleicacid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “bind,” “binds,” or “interacts with” means that one molecule recognizes and adheres to a particular second molecule in a sample or organism, but does not substantially recognize or adhere to other structurally unrelated molecules in the sample.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which includes an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, et al. Examples of viral vectors include, but are not limited to, adenoviral vectors, adeno-associated virus vectors, and retroviral vectors. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by attenuating the HIV virulence genes, for example, the genes env, vif, vpr, vpu, and nef are deleted making the vector biologically safe. In other embodiments of the present disclosure, a gamma retrovirus may be used as the transfecting agent.

Where used herein the terms “endogenous,” “native,” and “wild-type” refer to the typical form (genotype and/or phenotype) of a bacterium, gene, nucleic acid, protein, or peptide as it occurs in nature and/or is the most common form in a natural population, e.g., in its natural location in the organism or in the genome of the organism, and optionally with its own regulatory sequences, if present. In reference to a gene or nucleic acid, the term “mutation” refers to a gene or nucleic acid comprising an alteration in the wild type, such as but not limited to, a nucleotide deletion, insertion, and/or substitution. A mutation in a gene or nucleic acid generally results in either inactivation, decrease in expression or activity, increase in expression or activity, or another altered property of the gene or nucleic acid. In reference to a protein, the term “mutation” refers to protein comprising an alteration in the wild type, such as but not limited to, one or more amino acid deletions, insertions, and/or substitutions. A mutation in a protein may result in either inactivation, a decrease in activity or effect (e.g., binding), or an increase in activity or effect (e.g., binding), or another altered property or effect of the protein.

The term “plant” as used herein, includes a whole plant and any descendant, cell, tissue, or part of a plant. The term “plant parts” include any part(s) of a plant, including, for example and without limitation: a seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; corm; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein.

The term “nucleic acid” refers to a polynucleotide of high molecular weight which can be single-stranded or double-stranded, composed of monomers (nucleotides) containing a sugar, phosphate and a base which is either a purine or pyrimidine. Where used herein, the terms “polynucleotide,” “nucleic acid,” and “nucleic acid molecule” may be used interchangeably herein, and encompass a singular nucleic acid; plural nucleic acids; a nucleic acid fragment, variant, or derivative thereof; and nucleic acid constructs (e.g., messenger RNA (mRNA) and plasmid DNA (pDNA)). A polynucleotide or nucleic acid may contain the nucleotide sequence of a full-length cDNA sequence, or a fragment thereof, including untranslated 5′ and/or 3′ sequences and coding sequence(s). A polynucleotide or nucleic acid may be comprised of any polyribonucleotide or polydeoxyribonucleotide, which may include unmodified ribonucleotides or deoxyribonucleotides or modified ribonucleotides or deoxyribonucleotides. For example, a polynucleotide or nucleic acid may be comprised of single- and double-stranded DNA; DNA that is a mixture of single-stranded and double-stranded regions; single-stranded and double- stranded RNA; and RNA that is mixture of single-stranded and double-stranded regions. Hybrid molecules comprising DNA and RNA may be single-stranded, double-stranded, or a mixture of single-stranded and double-stranded regions. The foregoing terms also include chemically, enzymatically, and metabolically modified forms of a polynucleotide or nucleic acid. It is understood that a specific DNA refers also to the complement thereof, the sequence of which is determined according to the rules of deoxyribonucleotide base-pairing.

The term “expression” as used herein refers to the intracellular processes, including transcription and translation, by which a coding DNA molecule such as a structural gene produces RNA or a polypeptide.

Where used herein, the terms “transformation” or “genetic transformation” refer to a process of introducing a nucleic acid sequence or nucleic acid construct (e.g., a vector or expression cassette) into a cell or protoplast in which that exogenous nucleic acid (e.g., DNA) is incorporated into a chromosome or is capable of autonomous replication. When used in conjunction with a transgenic plant cell or transgenic plant, “obtaining” means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Where used herein, the term “heterologous” refers to a sequence which is not normally present in a given host genome in the genetic context in which the sequence is currently found. In this respect, the sequence may be native to the host genome, but is arranged in a different order with respect to other genetic sequences within the host (e.g., wild-type) sequence. For example, a regulatory sequence may be heterologous in that it is linked to a different coding sequence relative to the native regulatory sequence. That is, the transgenic plant may comprise a promoter of the same species, as long as the overall recombinant DNA construct containing the promoter is different from a DNA sequence of the wild-type of the species, for example either in the order of the DNA sequences of the recombinant DNA construct, or in the protein-encoding DNA sequence. For example, a rice promoter sequence may be used in a transgenic rice plant of the present disclosure, as long as the promoter sequence is operably linked to a gene of a different species or to a different sequence from a different rice strain, or is linked in a different order than in the wild-type rice plant.

Where used herein, the term “promoter” refers to a site on a DNA sequence or group of DNA sequences that provides an expression control element for a structural gene or sequence and to which RNA polymerase specifically binds and initiates RNA synthesis (transcription) of that gene or sequence.

Where used herein, the term “Ro transgenic plant” refers to a first generation plant that has been genetically transformed or has been regenerated from a plant cell or cells that have been genetically transformed.

Where used herein, the term “regeneration” refers to a process of growing a plant from a plant cell (e.g., plant protoplast, callus, or explant).

Where used herein, the term “transformation construct,” “nucleic acid construct,” or “expression cassette” refers to a chimeric DNA molecule which is designed for introduction into a host genome by genetic transformation. Transformation constructs may comprise nucleic acid sequences for directing expression of one or more exogenous genes of the construct.

Where used herein, the term “transformed cell” refers to a cell in which the DNA complement has been altered by the introduction of an exogenous DNA molecule into that cell.

Where used herein, the term “transgene” refers to a DNA sequence which has been incorporated into a host genome or is capable of autonomous replication in a host cell and is capable of causing the expression of one or more coding sequences. Transgenes provide the host cell, or plants regenerated therefrom, with a novel genotype and phenotype relative to the corresponding non-transformed cell or plant. Transgenes may be directly introduced into a plant by genetic transformation, or may be inherited from a plant of any previous generation which was transformed with the DNA segment.

Where used herein, the term “transgenic plant” refers to a plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Where used herein, the term “vector” refers to any means by which a DNA molecule may be introduced by transformation into a host cell. Aplasmid is an exemplary vector, as are expression cassettes isolated therefrom.

A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers. Unless otherwise indicated, a particular nucleic acid sequence of this invention also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985); and Rossolini, et al., Mol. Cell. Probes 8:91-98 (1994)). The term nucleic acid is used interchangeably with gene, cDNA, and mRNA encoded by a gene.

As used herein, the term “coding sequence” refers to a nucleic acid sequence that encodes a specific amino acid sequence. A “regulatory sequence” refers to a nucleotide sequence located upstream (e.g., 5′ non-coding sequences), within, or downstream (e.g., 3′ non-coding sequences) of a coding sequence, which influences the transcription, RNA processing or stability, or translation of the associated coding sequence. Regulatory sequences include, for example and without limitation: promoters; translation leader sequences; introns; polyadenylation recognition sequences; RNA processing sites; effector binding sites; and stem-loop structures.

The terms “open reading frame” and “ORF” refer to the amino acid sequence encoded between translation initiation and termination codons of a coding sequence. The terms “initiation codon” and “termination codon” refer to a unit of three adjacent nucleotides ('codon') in a coding sequence that specifies initiation and chain termination, respectively, of protein synthesis (mRNA translation).

As used herein, the term “codon degeneracy” refers to redundancy in the genetic code that permits variation of a particular nucleotide sequence without affecting the amino acid sequence of the encoded polypeptide. Since each codon consists of three nucleotides, and the nucleotides comprising DNA are restricted to four specific bases, there are 64 possible combinations of nucleotides, 61 of which encode amino acids (the remaining three codons encode signals ending translation). As a result, many amino acids are designated by more than one codon. For example, the amino acids alanine and proline are coded for by four triplets, serine and arginine by six, whereas tryptophan and methionine are coded by just one triplet. The “genetic code” that shows which codons encode which amino acids is commonly known in the art. The degeneracy therein allows for the bases of a DNA to vary over a wide range without altering the amino acid sequence of the proteins encoded by the DNA.

In some embodiments herein, when designing a coding sequence for improved expression in a host cell, the gene is designed such that the frequency of codon usage therein approaches the frequency of the preferred codon usage of the host cell. Accordingly, the term “codon-optimized” refers to genes or coding sequences of nucleic acids for transformation of various hosts, wherein codons in the gene or coding sequence has been altered to reflect the typical codon usage of the host organism without altering the polypeptide encoded by the nucleic acid. In examples, such optimization includes replacing at least one, more than one, a significant number, and/or all of the codons in the gene or coding sequence with one or more codons that are more frequently used in the genes of that organism.

Many organisms display a bias for use of particular codons to code for insertion of a particular amino acid in a growing peptide chain. Codon preference, or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored or designed for optimal gene expression in a given organism based on codon optimization.

Where used herein, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Sequences which are completely complementary are sequences which are entirely complementary throughout their entire length and have no base mismatches. Other sequences with lower degrees of homology also are contemplated.

A nucleic acid is said to be the “complement” of another nucleic acid molecule if the two nucleic acid molecules exhibit complete sequence complementarity. As used herein, nucleic acids are said to exhibit “complete complementarity” when every nucleotide of one of the molecules is complementary to a nucleotide of the other. Molecules that exhibit complete complementarity will generally hybridize to one another with sufficient stability to permit them to remain annealed to one another under conventional “high-stringency” conditions, for example as described elsewhere herein. As used herein, the term “polypeptide” includes a singular polypeptide, plural polypeptides, and fragments thereof. This term refers to a molecule comprised of monomers (amino acids) linearly linked by amide bonds (also known as peptide bonds). The term “polypeptide” refers to any chain or chains of two or more amino acids, and does not refer to a specific length or size of the product. Accordingly, peptides, dipeptides, tripeptides, oligopeptides, protein, amino acid chain, and any other term used to refer to a chain or chains of two or more amino acids, are included within the definition of “polypeptide,” and the foregoing terms are used interchangeably with “polypeptide” herein. A polypeptide may be isolated from a natural biological source or produced by recombinant technology, but a specific polypeptide is not necessarily translated from a specific nucleic acid. A polypeptide may be generated in any appropriate manner, including for example and without limitation, by chemical synthesis.

Where used herein, the term “heterologous” refers to a polynucleotide, gene or polypeptide that is not normally found at its location in the reference (host) organism. For example, a heterologous nucleic acid may be a nucleic acid that is normally found in the reference organism at a different genomic location. By way of further example, a heterologous nucleic acid may be a nucleic acid that is not normally found in the reference organism. A host organism comprising a heterologous polynucleotide, gene or polypeptide may be produced by introducing the heterologous polynucleotide, gene or polypeptide into the host organism. In particular examples, a heterologous polynucleotide comprises a native coding sequence, or portion thereof, that is reintroduced into a source organism in a form and in association with other sequences, that is different from the corresponding native polynucleotide.

In particular examples, a heterologous gene may comprise a native coding sequence, or portion thereof, that is reintroduced into a source organism in a form that is different from the corresponding native gene. For example, a heterologous gene may include a native coding sequence that is a portion of a chimeric gene including non-native regulatory regions that is reintroduced into the native host. In particular examples, a heterologous polypeptide is a native polypeptide that is reintroduced into a source organism in a form that is different from the corresponding native polypeptide.

A heterologous gene or polypeptide may be a gene or polypeptide that comprises a functional polypeptide or nucleic acid sequence encoding a functional polypeptide that is fused to another genes or polypeptide to produce a chimeric or fusion polypeptide, or a gene encoding the same. Genes and proteins of particular embodiments include specifically exemplified full-length sequences and portions, segments, fragments (including contiguous fragments and internal and/or terminal deletions compared to the full-length molecules), variants, mutants, chimerics, and fusions of these sequences.

As used herein, the term “modification” may refer to a change in a particular reference polynucleotide that results in reduced, substantially eliminated, or eliminated activity of a polypeptide encoded by the reference polynucleotide. A modification may also refer to a change in a reference polypeptide that results in reduced, substantially eliminated, or eliminated activity of the reference polypeptide. Alternatively, the term “modification” may refer to a change in a reference polynucleotide that results in increased or enhanced activity of a polypeptide encoded by the reference polynucleotide, as well as a change in a reference polypeptide that results in increased or enhanced activity of the reference polypeptide. Changes such as the foregoing may be made by any of several methods well-known in the art including, for example and without limitation: deleting a portion of the reference molecule; mutating the reference molecule (e.g., via spontaneous mutagenesis, via random mutagenesis, via mutagenesis caused by mutator genes, and via transposon mutagenesis); substituting a portion of the reference molecule; inserting an element into the reference molecule; down-regulating expression of the reference molecule; altering the cellular location of the reference molecule; altering the state of the reference molecule (e.g., via methylation of a reference polynucleotide, and via phosphorylation or ubiquitination of a reference polypeptide); removing a cofactor of the reference molecule; introduction of an antisense RNA/DNA targeting the reference molecule; introduction of an interfering RNA/DNA targeting the reference molecule; chemical modification of the reference molecule; covalent modification of the reference molecule; irradiation of the reference molecule with UV radiation or X-rays; homologous recombination that alters the reference molecule; mitotic recombination that alters the reference molecule; replacement of the promoter of the reference molecule; and/or combinations of any of the foregoing.

The terms “derivative,” “variant, and “mutant” as used herein, refer to a modification of an exemplary sequence herein. Such modifications include the substitution, insertion, and/or deletion of one or more bases of a coding sequence herein that preserve, slightly alter, or increase the function of the coding sequence in a crop species, and also include heterologous nucleic acids comprising a sequence having substantial sequence identity with an exemplary sequence herein, such that they may have the same, slightly altered, or increased functionality for use in expressing a transgene in a crop plant. A variant polypeptide may have substituted amino acids, and yet retain the functional activity of the reference polypeptide. “Variant” genes comprise a nucleotide sequence that encodes the same polypeptide as a reference gene or an equivalent polypeptide that has an activity equivalent or similar to the reference polypeptide.

In some embodiments of the present disclosure, variant genes can be used to produce variant proteins, and recombinant hosts can be used to produce the variant proteins. For example, variant genes and proteins can be constructed that comprise contiguous residues (amino acid or nucleotide) of any exemplified sequence herein. A variant gene or protein may have, for example and without limitation: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more contiguous residues (amino acids or nucleotides) that correspond to a segment (of the same size) in the exemplified sequence. Similarly sized segments, especially those for conserved regions, can also be used as probes and/or primers.

In some embodiments, a variant protein is “truncated” with respect to a reference, full-length protein. In some examples, a truncated protein retains the functional activity of the reference protein. By “truncated” protein, it is meant that a portion of a protein is cleaved off, for example, while the remaining truncated protein retains and exhibits the desired activity after cleavage. Cleavage may be achieved by any of various proteases. Furthermore, effectively cleaved proteins can be produced using molecular biology techniques, wherein the DNA bases encoding a portion of the protein are removed from the coding sequence, either through digestion with restriction endonucleases or other techniques available to the skilled artisan. A truncated protein may be expressed in a heterologous system, for example, E. coli, baculoviruses, plant-based viral systems, and yeast.

The term “operably linked” refers to an association of nucleic acid sequences on a single nucleic acid, wherein the function of one of the nucleic acid sequences is affected by another. For example, a promoter is operably linked with a coding sequence when the promoter is capable of effecting the expression of that coding sequence (e.g., the coding sequence is under the transcriptional control of the promoter). A coding sequence may be operably linked to a regulatory sequence in a sense or antisense orientation.

The term “promoter” refers to a DNA sequence capable of controlling the expression of a nucleic acid coding sequence or functional RNA by providing the recognition for RNA polymerase and other factors required for proper transcription. The controlled coding sequence is generally located downstream (3′) of the promoter sequence. A promoter may be derived in its entirety from a native gene, a promoter may be comprised of different elements derived from different promoters found in nature, or a promoter may even comprise synthetic DNA segments. It is understood by those skilled in the art that different promoters can direct the expression of a gene in different tissues or cell types, or at different stages of development, or in response to different environmental or physiological conditions. Examples of all of the foregoingpromoters are known and used in the art to control the expression of heterologous nucleic acids. Promoters that direct the expression of a gene in most cell types at most times are commonly referred to as “constitutive promoters.” Furthermore, while those in the art have (in many cases unsuccessfully) attempted to delineate the exact boundaries of regulatory sequences, it has come to be understood that DNA fragments of different lengths may have identical promoter activity. The promoter activity of a particular nucleic acid may be assayed using techniques familiar to those in the art. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

“Primary transformant” and “T0 generation” refer to transgenic plants that are of the same geneticgeneration as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The term “progeny” as used herein refers to the offspring of any generation of a parent plant prepared in accordance with the present disclosure, wherein the progeny comprises a selected DNA construct.

As used herein, the term “gene” refers to a nucleic acid that encodes a functional product (RNA or polypeptide/protein). A gene may include regulatory sequences preceding (5′ non-coding sequences) and/or following (3′ non-coding sequences) the sequence encoding the functional product. The terms “native gene” or “wild type gene” refer to a gene as found in nature. The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to atleast one of its other components.

The term “expression,” as used herein, may refer to the transcription and stable accumulation of sense (mRNA) or antisense RNA derived from a DNA. Expression may also refer to translation of mRNA into a polypeptide. As used herein, the term “overexpression” refers to expression that is higher than endogenous expression of the same gene or a related gene. Thus, a heterologous gene is “overexpressed” if its expression is higher than that of a comparable endogenous gene.

As used herein, the term “transformation” refers to the transfer and integration of a nucleic acid or fragment thereof into a host organism, resulting in genetically stable inheritance. Host organisms containing a transforming nucleic acid are referred to as “transgenic,” “recombinant,” or “transformed” organisms. Known methods of transformation include, for example: Agrobacterium tumefaciens- or A. rhizogenes-mediated transformation; calcium phosphate transformation; polybrene transformation; protoplast fusion; electroporation; ultrasonic methods (e.g., sonoporation); liposome transformation; microinjection; transformation with naked DNA; transformation with plasmid vectors; transformation with viral vectors; biolistic transformation (microparticle bombardment); silicon carbide WHISKERS-mediated transformation; aerosol beaming; and PEG-mediated transformation.

As used herein, the term “introduced” (in the context of introducing a nucleic acid into a cell) includes transformation of a cell, as well as crossing a plant comprising the nucleic acid with a secondplant, such that the second plant contains the nucleic acid, as may be performed utilizing conventional plant breeding techniques. Such breeding techniques are known in the art.

The terms “plasmid” and “vector,” as used herein, refer to an extra chromosomal element that may carry one or more gene(s) that are not part of the central metabolism of the cell. Plasmids and vectors typically are circular double-stranded DNA molecules. However, plasmids and vectors may be linear or circular nucleic acids, of a single- or double-stranded DNA or RNA, and may be derived from any source, in which a number of nucleotide sequences have been joined or recombined into a unique construction that is capable of introducing a promoter fragment and a coding DNA sequence along with any appropriate 3′ untranslated sequence into a cell. In examples, plasmids and vectors may comprise autonomously replicating sequences, genome integrating sequences, and/or phage or nucleotide sequences.

The selection of a promoter used to direct expression of a nucleic acid herein depends on the particular application. A number of promoters that direct expression of a gene in a plant may be employed in embodiments herein. Such promoters can be selected from constitutive, chemically-regulated, inducible, tissue-specific, and seed-preferred promoters. For example, a strong constitutive promoter suited to the host cell may be used for expression and purification of DGT-28 proteins. Non-limiting examples of plant promoters include promoter sequences derived from A. thaliana ubiquitin-10 (ubi-10) (Callis, et al., 1990, J. Biol. Chem., 265:12486-12493); A. tumefaciens mannopine synthase (Amas) (U.S. Pat. No. 6,730,824); and/or Cassava Vein Mosaic Virus (CsVMV) (Verdaguer et al., 1996, Plant Molecular Biology 31:1129-1139).

Constitutive promoters include, for example, the core Cauliflower Mosaic Virus 35S promoter (Odell etal. (1985) Nature 313:810-812); Rice Actin promoter (McElroy et al. (1990) Plant Cell 2:163-171); Maize ubiquitin promoter (U.S. Pat. No. 5,510,474; Christensen et al. (1989) Plant Mol. Biol. 12:619-632 and Christensen et al. (1992) Plant Mol. Biol. 18:675-689); pEMU promoter (Last et al. (1991) Theor. Appl. Genet. 81:581-588); ALS promoter (U.S. Pat. No. 5,659,026); Maize Histone promoter (Chaboute et al. Plant Molecular Biology, 8:179-191 (1987)); and the like.

The range of available plant compatible promoters includes tissue specific and inducible promoters. An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription is present in an inactive form, which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. A plant cell containing an inducible regulatory element may be exposed to an inducer by externally applying the inducer to the cell or plant such as by spraying, watering, heating or similar methods.

Any inducible promoter can be used in embodiments herein (e.g., see Ward et al. Plant Mol. Biol. 22: 361-366 (1993)). Inducible promoters include, for example and without limitation: ecdysone receptor promoters (U.S. Pat. No. 6,504,082); promoters from the ACE1 system which respond to copper (Mett et al. PNAS 90: 4567- 4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)); Tet repressor from Tn10 (Gatz et al., Mol. Gen. Genet. 227: 229-237 (1991); promoters from a steroid hormone gene, the transcriptional activity of which is induced by a glucocorticosteroid hormone, Schena et al., Proc. Natl. Acad. Sci. U.S.A. 88: 10421 (1991) and McNellis et al., (1998) Plant J. 14(2):247-257; the maize GST promoter, which is activated by hydrophobic electrophilic compounds that are used as pre-emergent herbicides (see U.S. Pat. No. 5,965,387 and International Patent Application, Publication No. WO 93/001294); and the tobacco PR-la promoter, which is activated by salicylic acid (see Ono S, Kusama M, Ogura R, Hiratsuka K., “Evaluation of the Use of the Tobacco PR-la Promoter to Monitor Defense Gene Expression by the Luciferase Bioluminescence Reporter System,” Biosci Biotechnol Biochem. 2011 Sep. 23; 75(9):1796-800). Other chemical-regulated promoters of interest include tetracycline-inducible and tetracycline-repressible promoters (see, for example, Gatz et al., (1991) Mol. Gen. Genet. 227:229-237, and U.S. Pat. Nos. 5,814,618 and 5,789,156).

Other regulatable promoters of interest include a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; the light-inducible promoter derived from the pea rbcS gene or pea psaDb gene (Yamamoto et al. (1997) Plant J. 12(2):255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90(20):9586-9590; Orozco et al. (1993) Plant Mol. Bio. 23(6):1129-1138); a plant hormone inducible regulatory element (Yamaguchi-Shinozaki et al., Plant Mol. Biol. 15:905, 1990; Kares et al., Plant Mol. Biol. 15:225, 1990), and the like. An inducible regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genet. 227:229-237, 1991).

Stress inducible promoters include salt/water stress-inducible promoters such as PSCS (Zang et al. (1997) Plant Sciences 129:81-89); cold-inducible promoters, such as cor15a (Hajela et al. (1990) Plant Physiol. 93:1246-1252), corl5b (Wilhelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), and ci21A (Schneider et al. (1997) Plant Physiol. 113:335-45); drought-inducible promoters, such as Trg-31 (Chaudhary et al. (1996) Plant Mol. Biol. 30:1247-57) and rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338); and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331- 340).

Tissue-preferred promoters may be utilized to target enhanced transcription and/or expression within a particular plant tissue. Examples of these types of promoters include seed-preferred expression, such as that provided by the phaseolin promoter (Bustos et al. 1989. The Plant Cell Vol. 1, 839-853), and the maize globulin-1 gene, Belanger, et al. 1991 Genetics 129:863-972. For dicots, seed-preferred promoters include, but are not limited to, bean beta-phaseolin, napin, beta-conglycinin, soybean lectin, cruciferin, and the like. For monocots, seed-preferred promoters include, but are not limited to, maize 15 kDa zein, 22 kDa zein, 27 kDa zein, gamma-zein, waxy, shrunken 1, shrunken 2, globulin 1, etc. Seed-preferred promoters also include those promoters that direct gene expression predominantly to specific tissues within the seed such as, for example, the endosperm-preferred promoter of gamma-zein, the cryptic promoter from tobacco (Fobert et al. 1994. T-DNA tagging of a seed coat-specific cryptic promoter in tobacco. Plant J. 4: 567-577), the P- gene promoter from corn (Chopra et al. 1996. Alleles of the maize P gene with distinct tissue specificities encode Myb-homologous proteins with C-terminal replacements. Plant Cell 7:1149-1158, Erratum in Plant Cell. 1997, 1:109), the globulin-1 promoter from corn (Belenger and Kriz. 1991. Molecular basis for Allelic Polymorphism of the maize Globulin-1 gene. Genetics 129: 863-972), and promoters that direct expression to the seed coat or hull of corn kernels, for example the pericarp-specific glutamine synthetase promoter (Muhitch et al., 2002. Isolation of a Promoter Sequence From the Glutamine Synthetase)-2 Gene Capable of Conferring Tissue-Specific Gene Expression in Transgenic Maize. Plant Science163:865-872).

In addition to the promoter, an expression vector typically contains a transcription unit or expression cassette that contains all the additional elements required for the expression of the nucleic acid in host cells, either prokaryotic or eukaryotic. A typical expression cassette thus contains a promoter operably-linked, e.g., to a nucleic acid sequence encoding the protein, and signals required, e.g., for efficient polyadenylation of the transcript, transcriptional termination, ribosome binding sites, or translation termination. Additionalelements of the cassette may include, e.g., enhancers and heterologous splicing signals.

Other components of the vector may be included, also depending upon intended use of the gene. Examples include selectable markers, targeting or regulatory sequences, transit peptide sequences such as the optimized transit peptide sequence (see U.S. Pat. No. 5,510,471) stabilizing sequences such as RB7 MAR (see Thompson and Myatt, (1997) Plant Mol. Biol., 34: 687-692 and WO9727207) or leader sequences, introns etc. General descriptions and examples of plant expression vectors and reporter genes can be found in Gruber, et al., “Vectors for Plant Transformation” in Methods in Plant Molecular Biology and Biotechnology, Glick et al. eds; CRC Press pp. 89-119 (1993).

The selection of an appropriate expression vector will depend upon the host and the method of introducing the expression vector into the host. The expression cassette may include, at the 3′ terminus of a heterologous nucleotide sequence of interest, a transcriptional and translational termination region functional in plants. The termination region can be native with the DNA sequence of interest or can be derived from another source. Convenient termination regions are available from the Ti-plasmid of A. tumefaciens, such as the octopine synthase and nopaline synthase (nos) termination regions (Depicker et al., Mol. and Appl. Genet. 1:561-573 (1982) and Shaw et al. (1984) Nucleic Acids Research vol. 12, No. 20 pp 7831-7846 (nos)); see also Guerineau et al. Mol. Gen. Genet. 262:141-144 (1991); Proudfoot, Cell 64:671-674 (1991); Sanfacon et al. Genes Dev. 5:141-149 (1991); Mogen et al. Plant Cell 2:1261-1272 (1990); Munroe et al. Gene 91:151-158 (1990); Ballas et al. Nucleic Acids Res. 17:7891-7903 (1989); Joshi et al. Nucleic Acid Res. 15:9627-9639 (1987).

An expression cassette may contain a 5′ leader sequence. Such leader sequences can act to enhance translation. Translation leaders are known in the art and include by way of example, picornavirus leaders, EMCV leader (Encephalomyocarditis 5′ noncoding region), Elroy-Stein et al. Proc. Nat. Acad. Sci. USA 86:6126-6130 (1989); potyvirus leaders, for example, TEV leader (Tobacco Etch Virus) Carrington and Freed Journal of Virology, 64:1590-1597 (1990), MDMV leader (Maize Dwarf Mosaic Virus), Allison et al., Virology 154:9-20 (1986); human immunoglobulin heavy-chain binding protein (BiP), Macejak et al. Nature 353:90-94 (1991); untranslated leader from the coat protein mRNA of alfalfa mosaic virus (AMV RNA 4), Jobling et al. Nature 325:622-625 (1987); Tobacco mosaic virus leader (TMV), Gallie et al. (1989) Molecular Biology of RNA, pages 237-256; and maize chlorotic mottle virus leader (MCMV) Lommel et al. Virology 81:382-385 (1991). See also Della-Cioppa et al. Plant Physiology 84:965-968 (1987).

The construct may also contain sequences that enhance translation and/or mRNA stability such as introns. An example of one such intron is the first intron of gene II of the histone H3.III variant of Arabidopsis thaliana. Chaubet et al. Journal of Molecular Biology, 225:569-574 (1992).

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase and Helianthus annuus (see Lebrun et al. U.S. Pat. No. 5,510,417), Zea mays Brittle-1 chloroplast transit peptide (Nelson et al. Plant Physiol 117(4):1235-1252 (1998); Sullivan et al. Plant Cell 3(12):1337-48; Sullivan etal., Planta (1995) 196(3):477-84; Sullivan et al., J. Biol. Chem. (1992) 267(26):18999-9004) and the like. In addition, chimeric chloroplast transit peptides are known in the art, such as the Optimized Transit Peptide (see, U.S. Pat. No. 5,510,471). Additional chloroplast transit peptides have been described previously in U.S. Pat. Nos. 5,717,084 and 5,728,925. One skilled in the art will readily appreciate the many options available in expressing a product to a particular organelle. For example, the barley alpha amylase sequence is often used to direct expression to the endoplasmic reticulum. Rogers, J. Biol. Chem. 260:3731-3738 (1985).

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post- translational modifications. Additionally, the promoter sequence might be genetically engineered to improve the level of expression as compared to the native promoter. Recombinant techniques useful for controlling the expression of nucleic acid molecules include, but are not limited to, stable integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno or Kozak sequences), modification of nucleic acid molecules to correspond to the codon usage of the host cell, and deletion of sequences that destabilize transcripts.

Reporter or marker genes for selection of transformed cells or tissues or plant parts or plants may beincluded in the transformation vectors. Examples of selectable markers include those that confer resistance to anti- metabolites such as herbicides or antibiotics, for example, dihydrofolate reductase, which confers resistance to methotrexate (Reiss, Plant Physiol. (Life Sci. Adv.) 13:143-149, 1994; see also Herrera Estrella et al., Nature 303:209-213, 1983; Meijer et al., Plant Mol. Biol. 16:807-820, 1991); neomycin phosphotransferase, which confers resistance to the aminoglycosides neomycin, kanamycin and paromycin (Herrera-Estrella, EMBO J. 2:987-995, 1983 and Fraley et al. Proc. Natl. Acad. Sci USA 80:4803 (1983)); hygromycin phosphotransferase, which confers resistance to hygromycin (Marsh, Gene 32:481-485, 1984; see also Waldron et al., Plant Mol. Biol. 5:103-108, 1985; Zhijian et al., Plant Science 108:219-227, 1995); trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995).

In some embodiments, a cell and/or organism (e.g., a plant cell or plant) is provided that comprises a heterologous polypeptide having at least 90% identity to a polypeptide sequence disclosed herein. In particular embodiments, a cell and/or organism is provided that comprises a heterologous nucleic acid encoding a polypeptide having at least 90% identity to said sequence.

A plant cell, plant part, and/or plant of the disclosure may be genetically modified to comprise a heterologous polypeptide and/or heterologous nucleic acid by any of several methods of introducing a heterologous molecule known in the art. In particular embodiments herein, a heterologous molecule is introduced into a plant cell, plant part, and/or plant by a method selected from,for example and without limitation: transformation and selective breeding (e.g., backcross breeding).

Nucleic acids introduced into a plant cell may be used to confer desired traits on essentially any plant. A wide variety of plants and plant cell systems may be engineered for the desired physiological and agronomic characteristics described herein. Numerous methods for plant transformation have been developed, including biological and physical transformation protocols for dicotyledonous plants, as well as monocotyledenous plants (See, e.g., Goto-Fumiyuki et al. (1999) Nat. Biotechnol. 17:282-6; Miki et al. (1993) Methods in Plant Molecular Biology and Biotechnology (Glick, B. R. and Thompson, J. E., Eds.), CRC Press, Inc., Boca Raton, Fla., pp. 67-88). In addition, vectors and in vitro culture methods for plant cell and tissue transformation and regeneration of plants are described, for example, in Gruber et al. (1993), supra, at pp. 89-119.

Plant transformation techniques available for introducing a nucleic acid into a plant host cell include, for example and without limitation: transformation with disarmed T-DNA using Agrobacterium tumefaciens or A. rhizogenes as the transformation agent; calcium phosphate transfection; polybrene transformation; protoplast fusion; electroporation (D'Halluin et al. (1992) Plant Cell 4:1495-505); ultrasonic methods (e.g., sonoporation); liposome transformation; microinjection; contact with naked DNA; contact with plasmid vectors; contact with viral vectors; biolistics (e.g., DNA particle bombardment (see, e.g., Klein et al. (1987) Nature 327:70-3) and microparticle bombardment (Sanford et al. (1987) Part. Sci. Technol. 5:27; Sanford (1988) Trends Biotech. 6:299, Sanford (1990) Physiol. Plant 79:206; and Klein et al. (1992) Biotechnology 10:268); silicon carbide WHISKERS-mediated transformation (Kaeppler et al. (1990) Plant Cell Rep. 9:415- 8); nanoparticle transformation (see, e.g., U.S. Patent Publication No. US2009/0104700A1); aerosol beaming; and polyethylene glycol (PEG)-mediated uptake. In specific examples, a heterologous nucleic acid may be introduced directly into the genomic DNA of a plant cell.

A widely utilized method for introducing an expression vector into a plant is based on the natural transformation system of Agrobacterium. Horsch et al. (1985) Science 227:1229. A. tumefaciens and A. rhizogenes are plant pathogenic soil bacteria known to be useful to genetically transform plant cells. The Ti and Ri plasmids of A. tumefaciens and A. rhizogenes, respectively, carry genes responsible for genetic transformation of the plant. Kado (1991) Crit. Rev. Plant. Sci. 10:1. Details regarding Agrobacterium vector systems and methods for Agrobacterium-mediated gene transfer are also available in, for example, Gruberet al., supra, Mild et al., supra, Moloney et al. (1989) Plant Cell Reports 8:238, and U.S. Pat. Nos. 4,940,838 and 5,464,763.

If Agrobacterium is used for the transformation, the DNA to be inserted typically is cloned into special plasmids; either into an intermediate vector or a binary vector. Intermediate vectors cannot replicate themselves in Agrobacterium. The intermediate vector may be transferred into A. tumefaciens by means of a helper plasmid (conjugation). The Japan Tobacco Superbinary system is an example of such a system (reviewed by Komari et al. (2006) Methods in Molecular Biology (K. Wang, ed.) No. 343; Agrobacterium Protocols, 2.sup.nd Edition, Vol. 1, Humana Press Inc., Totowa, N.J., pp. 15-41; and Komori et al. (2007) Plant Physiol. 145:1155-60). Binary vectors can replicate themselves both in E. coli and in Agrobacterium.

Binary vectors comprise a selection marker gene and a linker or polylinker which are framed by the right and left T-DNA border regions. They can be transformed directly into Agrobacterium (Holsters, 1978). The Agrobacterium comprises a plasmid carrying a vir region. The Ti or Ri plasmid also comprises the vir region necessary for the transfer of the T-DNA. The vir region is necessary for the transfer of the T-DNA into the plant cell. Additional T-DNA may be contained. The virulence functions of the Agrobacterium tumefaciens host will direct the insertion of a T-strand containing the construct and adjacent marker into the plant cell DNA when the cell is infected by the bacteria using a binary T DNA vector (Bevan (1984) Nuc. Acid Res. 12:8711-21) or the co-cultivation procedure (Horsch et al. (1985) Science 227:1229-31). Generally, the Agrobacterium transformation system is used to engineer dicotyledonous plants. Bevan et al. (1982) Ann. Rev. Genet 16:357-84; Rogers et al. (1986) Methods Enzymol. 118:627-41. The Agrobacterium transformation system may also be used to transform, as well as transfer, nucleic acids to monocotyledonous plants and plant cells. See U.S. Pat. No. 5,591,616; Hernalsteen et al. (1984) EMBO J 3:3039-41; Hooykass-Van Slogteren et al. (1984) Nature 311:763-4; Grimsley et al. (1987) Nature 325:1677-9; Boulton et al. (1989) Plant Mol. Biol. 12:31-40; and Gould et al. (1991) Plant Physiol. 95:426-34.

Agrobacterium-mediated transfer is a widely applicable system for introducing genes into plant cellsbecause the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described in U.S. Pat. No. 5,563,055, which is specifically incorporated herein by reference in its entirety. Agrobacterium-mediated transformation is most efficient in dicotyledonous plants and is often the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. An example of how Agrobacterium-mediated transformation techniques can be applied to rice are shown in U.S. Pat. No. 5,591,616, specifically incorporated herein by reference in its entirety. One also may employ protoplasts for electroporation transformation of plants.

Another method for delivering transforming DNA segments to plant cells is microprojectile bombardment (e.g., see U.S. Pat. Nos. 5,550,318; 5,538,880; 5,610,042; and PCT Application WO 94/09699; each of which is specifically incorporated herein by reference in its entirety. In this method, particles may be coated with nucleic acids and delivered into cells by a propelling force. Exemplary particles include those comprised of tungsten, platinum, and gold. For the bombardment, cells in suspension can be concentrated on filters or solid culture medium. Alternatively, immature embryos or other target cells may be arranged on solid culture medium. The cells to be bombarded are positioned at an appropriate distance below a macroprojectile stopping plate. Non-limiting examples of species for which have been transformed by microprojectile bombardment include monocot species such as maize, barley, wheat, rice, oat, rye, sugarcane, and sorghum, as well as a number of dicots including tobacco, soybean, sunflower, peanut, cotton, tomato, and legumes.

For example, in an embodiment of creation of transgenic switchgrass plants using particle bombardment, callus was bombarded with a plasmid carrying a sgfp (green fluorescent protein) gene and a bar (bialaphos and Basta tolerance) gene under control of a rice actin promoter and maize ubiquitin promoter respectively. Plants regenerated from bombarded callus were Basta tolerant and expressed GFP. These primary transformants were then crossed with non-transgenic control plants, and Basta tolerance was observed in progeny plants, demonstrating inheritance of the bar gene.

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

After effecting delivery of exogenous DNA to recipient cells, the next steps generally concern identifying the transformed cells for further culturing and plant regeneration. In order to improve the ability to identify transformants, a selectable or screenable marker gene may be used with a transformation vector prepared in accordance with the present disclosure. In this case, one would then generally assay the potentially transformed cell population by exposing the cells to a selective agent or agents, or one would screen the cells for the desired marker gene trait. In order to identify cells which received and integrated the DNA construct, one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase. Screenable markers may be used, such as the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein. Cells that survive the exposure to the selective agent, or cells that have been scored positive in a screening assay, may be cultured in media that supports regeneration of plants.

Tissue may be maintained on a basic media with growth regulators until sufficient tissue is available to begin plant regeneration efforts, or following repeated rounds of manual selection, until the morphology of the tissue is suitable for regeneration, e.g., at least 2 weeks, then transferred to media conducive to maturation of embryoids. Cultures may be transferred every 2 weeks for example. Shoot development will signal the time to transfer to medium lacking growth regulators. The transformed cells, identified by selection or screening and cultured in an appropriate medium that supports regeneration, will then be allowed to mature into plants. Developing plantlets are transferred to soilless plant growth mix, and hardened, e.g., in an environmentally controlled chamber. Plants may be matured in a growth chamber or greenhouse. Plants can be regenerated from about 6 weeks to 10 months after a transformant is identified, depending on the initial tissue. During regeneration, cells are grown on solid media in tissue culture vessels. After the regenerating plants have reached the stage of shoot and root development, they may be transferred to a greenhouse for further growth and testing.

In addition to direct transformation of a particular plant genotype with a construct described herein, transgenic plants may be made by crossing a plant having a selected DNA of the present disclosure to a second plant lacking the construct. For example, a transgenic event can be introduced into a particular plant variety by crossing, without the need for ever directly transforming a plant of that given variety.

Therefore, the present disclosure, in certain embodiments not only encompasses a plant directly transformed or regenerated fromcells which have been transformed, but also the progeny of such plants.

The genetic manipulations of a recombinant host herein may be performed using standard genetic techniques and screening, and may be carried out in any host cell that is suitable to genetic manipulation. In some embodiments, a recombinant host cell may be any organism or microorganism host suitable for genetic modification and/or recombinant gene expression. In some embodiments, a recombinant host may be a plant. Standard recombinant DNA and molecular cloning techniques used here are well-known in the art and are described in, for example and without limitation: Sambrook et al. (1989), supra; Silhavy et al. (1984) Experiments with Gene Fusions, Cold Spring Harbor Laboratory Press, Cold Spring Harbor, N.Y.; and Ausubel et al. (1987) Current Protocols in Molecular Biology, Greene Publishing Assoc. and Wiley-Interscience, New York, N.Y.

A transformed plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the beta-glucuronidase, luciferase, or gfp genes) that may be present on the recombinant nucleic acid constructs. Such selection and screening methodologies are well known to those skilled in the art.

A transgenic plant containing a heterologous molecule herein can be produced through selective breeding, for example, by sexually crossing a first parental plant comprising the molecule, and a second parental plant, thereby producing a plurality of first progeny plants. A first progeny plant may then be selected that is resistant to a selectable marker (e.g., glyphosate, resistance to which may be conferred upon the progeny plant by the heterologous molecule herein). The first progeny plant may then by selfed, thereby producing a plurality of second progeny plants. Then, a second progeny plant may be selected that is resistant to the selectable marker. These steps can further include the back-crossing of the first progeny plant or the second progeny plant to the second parental plant or a third parental plant.

It is also to be understood that two different transgenic plants can also be mated to produce offspring that contain two independently segregating, added, exogenous genes. Selfing of appropriate progeny can produce plants that are homozygous for both added, exogenous genes. Back-crossing to a parental plant and out- crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The resulting plant is expected to have the attributes of the recurrentparent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

A nucleic acid may also be introduced into a predetermined area of the plant genome through homologous recombination. Methods to stably integrate a polynucleotide sequence within a specific chromosomal site of a plant cell via homologous recombination have been described within the art. For instance, site specific integration as described in US Patent Application Publication No. 2009/0111188 A1 involves the use of recombinases or integrases to mediate the introduction of a donor polynucleotide sequence into a chromosomal target. In addition, International Patent Application No. WO 2008/021207, describes zinc finger mediated-homologous recombination to stably integrate one or more donor polynucleotide sequences within specific locations of the genome. The use of recombinases such as FLP/FRT as described in U.S. Pat. No. 6,720,475, or CRE/LOX as described in U.S. Pat. No. 5,658,772, can be utilized to stably integrate a polynucleotide sequence into a specific chromosomal site. Finally, the use of meganucleases for targeting donor polynucleotides into a specific chromosomal location was described in Puchta et al., PNAS USA 93 (1996) pp. 5055-5060).

In some embodiments, a heterologous nucleic acid may be optionally combined with another nucleic acid in the host cell and/or organism. For example, in certain embodiments, the heterologous nucleic acid encoding a polypeptide may be combined or “stacked” with another that provides additional resistance or tolerance to glyphosate or another herbicide, and/or another that provides resistance to select insects or diseases and/or nutritional enhancements, and/or improved agronomic characteristics, and/or another that provides proteins or other products useful in feed, food, industrial, pharmaceutical or other uses. The “stacking” of two or more nucleic acid sequences of interest within a plant genome may be accomplished, for example, via conventional plant breeding using two or more events, transformation of a plant with a construct(s) that contain the nucleic acids, re-transformation of a transgenic plant, or addition of new traits through targeted integration via homologous recombination.

Examples of such heterologous genes or coding Sequence include those that confer resistance to pests or disease, including but not limited to, genes for (a) plant disease resistance, including tomato Cf-9 gene for resistance to Cladosporium fulvum (Jones et al., 1994 Science 266:789), tomato Pto gene, which encodes a protein kinase, for resistance to Pseudomonas syringae pv. tomato (Martin et al., 1993 Science 262:1432), and Arabidopsis RSSP2 gene for resistance to Pseudomonas syringae (Mindrinos et al., 1994 Cell 78:1089), (b) Bacillus thuringiensis protein, a derivative thereof or a synthetic polypeptide modeled thereon, such as, a nucleotide sequence of a Bt delta-endotoxin gene (Geiser et al., 1986 Gene 48:109), and a vegetative insecticidal (VIP) gene (see, e.g., Estruch et al. (1996) Proc. Natl. Acad. Sci. 93:5389-94), and genes encoding delta-endotoxin (e.g., American Type Culture Collection (ATCC) accession numbers 40098, 67136, 31995 and 31998), (c) a lectin, such as, nucleotide sequences of several Clivia miniata mannose-binding lectin genes (Van Damme et al., 1994 Plant Molec. Biol. 24:825), (d) vitamin binding proteins, such as avidin and avidin homologs which are useful as larvicides against insect pests (see U.S. Pat. No. 5,659,026), (e) enzyme inhibitors, e.g., a protease inhibitor or an amylase inhibitor, e.g., a rice cysteine proteinase inhibitor (Abe et al., 1987 J. Biol. Chem. 262:16793), a tobacco proteinase inhibitorl (Huub et al., 1993 Plant Molec. Biol. 21:985), and an alpha-amylase inhibitor (Sumitani et al., 1993 Biosci. Biotech. Biochem. 57:1243), (f) insect-specific hormones or pheromones such as an ecdysteroid and juvenile hormone a variant thereof, a mimetic based thereon, or an antagonist or agonist thereof, such as baculovirus expression of cloned juvenile hormone esterase, an inactivator of juvenile hormone (Hammock et al., 1990 Nature 344:458), (g) insect-specific peptides or neuropeptides which, upon expression, disrupt the physiology of the affected pest (J. Biol. Chem. 269:9), e.g., an insect diuretic hormone receptor (Regan, 1994), an allostatin identified in Diploptera punctata (Pratt, 1989), and insect-specific, paralytic neurotoxins (U.S. Pat. No. 5,266,361), (h) insect-specific venom produced in nature by a snake, a wasp, etc., such as a scorpion insecto toxic peptide (Pang, 1992 Gene 116:165), (i) an enzyme responsible for a hyperaccumulation of monoterpene, a sesquiterpene, a steroid, hydroxamic acid, a phenylpropanoid derivative or another non-protein molecule with insecticidal activity, (j) an enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic, e.g., a callas gene (PCT published application W093/02197), chitinase-encoding sequences (which can be obtained, for example, from the ATCC under accession numbers 3999637 and 67152), tobacco hookworm chitinase (Kramer et al., 1993 Insect Molec. Biol. 23:691), and parsley ubi4-2 polyubiquitin gene (Kawalleck et al., 1993 Plant Molec. Biol. 21:673), (k) a molecule that stimulates signal transduction, e.g., nucleotide sequences for mung bean calmodulin cDNA clones (Botella et al., 1994 Plant Molec. Biol. 24:757) and maize calmodulin cDNA clone (Griess et al., 1994 Plant Physiol. 104:1467), (1) hydrophobic moment peptides, e.g., U.S. Pat. Nos. 5,659,026 and 5,607,914, (m) membrane permeases, channel formers or channel blockers, such as a cecropin-.beta. lytic peptide analog (Jaynes et al., 1993 Plant Sci. 89:43) which renders transgenic tobacco plants resistant to Pseudomonas solanacearum, (n) viral-invasive proteins or a complex toxins derived therefrom, e.g., whereby the accumulation of viral coat proteins in transformed plant cells impart resistance to viral infection and/or disease development effected by the virus from which the coat protein gene is derived, as well as by related viruses, e.g., coat protein- mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus, e.g., see, for example, Beachy et al. (1990) Ann. Rev. Phytopathol. 28:451, (o) an insect-specific antibody or an immunotoxin derived therefrom, e.g., Taylor et al. (1994) Abstract #497, Seventh Int'l. Symposium on Molecular Plant-Microbe Interactions shows enzymatic inactivation in transgenic tobacco via production of single-chain antibody fragments, (p) virus-specific antibodies, e.g., Tavladoraki et al. (1993) Nature 266:469, which shows that transgenic plants expressing recombinant antibody genes are protected from virus attack, (q) developmental-arrestive proteins produced in nature by a pathogen or a parasite, e.g., fungal endoalpha-1,4-D polygalacturonases facilitate fungal colonization and plant nutrient release by solubilizing plant cell wall homo-alpha-1,4-D-galacturonase (Lamb et al., 1992) Bio/Technology 10:1436 and Toubart et al. (1992 Plant J. 2:367), and (r) a developmental-arrestive protein produced in nature by a plant, such as the barley ribosome-inactivating gene that provides an increased resistance to fungal disease, e.g., Longemann et al., 1992 Bio/Technology 10:3305).

In certain embodiments, the transgenic plant of the present disclosure is a forage plant, a biofuel crop, or a cereal crop. Where used herein, the term “forage crops” refers to crops including grasses and legumes used as fodder or silage for livestock production. Examples of such plants that can be modified as described herein to decrease lignin production include, but are not limited to, switchgrass (Panicum virgatum), giant reed (Arundo donax), reed canarygrass (Phalaris arundinacea), MiscanthusXgiganteus, Miscanthus sp., Sericea lespedeza (Lespedeza cuneata), corn, sugarcane, sorghum, millet, rye, ryegrass (Lolium multiflorum, Lolium sp.), timothy, Kochia (Kochia scoparia), soybean, alfalfa, clover, sunn hemp, kenaf, bahiagrass, bermudagrass, dallisgrass, pangolagrass, big bluestem, indiangrass, fescue (Festuca sp.), centipede grass (Eremochloa ophiuroides), Dactylis sp., purple false brome (Brachypodium distachyon), smooth bromegrass, orchardgrass, Kentucky bluegrass, poplar, rice, cotton, red sage (Salvia miltiorrhiza), apple, common grape (Vitis vinifera), castor bean (Ricinus communis), hops (Humulus lupulus), Dahlia, orchids, mustard (Brassica rapa), kudzu (Pueraria lobata), wheat, eucalyptus, alder, and cedar.

In certain embodiments, the transgenic plant comprising the nucleic acid construct is a first generation (Ro) transgenic plant, or a progeny of any generation of said Ro transgenic plant, wherein the transgenic plant has inherited the nucleic acid construct. In certain embodiments, the heterologous promoter sequence is an organelle-specific, inducible, tissue-specific, constitutive, cell-specific, seed specific, and/or germination-specific promoter.

In certain aspects, the transgenic plants of the present disclosure can be used as a biomass source for production of ethanol. The overall process for the production of ethanol from biomass typically involves two steps: saccharification and fermentation. First, saccharification produces fermentable sugars from the cellulose and hemicellulose in the lignocellulosic biomass. Second, those sugars are then fermented to produce ethanol. Raw biomass may be pretreated to increase porosity, hydrolyze hemicellulose, remove lignin and reduce cellulose crystallinity, all in order to improve recovery of fermentable sugars from the cellulose polymer. As a preliminary step in pretreatment, the lignocellulosic material may be chipped or ground. The size of the biomass particles after chipping or grinding is typically between 0.2 and 30 mm. After chipping a number of other pretreatment options may be used to further prepare the biomass for saccharification and fermentation, including steam explosion, ammonia fiber explosion, acid hydrolysis, alkaline hydrolysis, oxidative delignification, organosolv process, and biological pretreatment.

After pretreatment, the cellulose in the lignocellulosic biomass may be hydrolyzed with cellulase enzymes. Cellulase catalyzes the breakdown of cellulose to release glucose which can then be fermented intoethanol. Bacteria and fungi produce cellulases suitable for use in ethanol production. Cellulases are usually actually a mixture of several different specific activities. First, endoglucanases create free chain ends of the cellulose fiber. Exoglucanases remove cellobiose units from the free chain ends and beta-glucosidase hydrolyzes cellobiose to produce free glucose. Aside from enzymatic hydrolysis, cellulose may also be hydrolyzed with weak acids or hydrochloric acid.

Once fermentable sugars have been produced from the lignocellulosic biomass, those sugars may be used to produce ethanol via fermentation. For maximum efficiencies, both pentose sugars from the hemicellulose fraction of the lignocellulosic material (e.g., xylose) and hexose sugars from the cellulose fraction (e.g., glucose) should be utilized. Saccharomyces cerevisiae (yeast) are widely used for fermentation of hexose sugars. Pentose sugars, released from the hemicellulose portion of the biomass, may be fermented using genetically engineered bacteria. Simultaneous saccharification and fermentation (SSF) is an alternative to the above described separate saccharification and fermentation steps. In addition to increased cellulose utilization, SSF also eliminates the need for a separate vessel and processing step. A typical temperature for SSF is around 38 deg. C. and can proceed up to 5 to 7 days for example. The final step for production of ethanol is distillation. The fermentation or SSF product is distilled using conventional methods producing ethanol, for instance 95% ethanol.

Recombinant nucleic acid constructs of the present disclosure may further comprise one or more additional DNA sequences that downregulate lignin, hemicellulose, or cellulose biosynthesis. For example, in certain embodiments, the additional DNA sequence may downregulate a lignin biosynthesis gene, for example 4-coumarate 3-hydroxylase (C3H), phenylalanine ammonia- lyase (PAL), cinnamate 4-hydroxylase (C4H), hydroxycinnamoyl transferase (HCT), caffeic acid 0-methyltransferase (COMT), caffeoyl CoA 3-O-methyltransferase (CCoAOMT), ferulate 5-hydroxylase (F5H), cinnamyl alcohol dehydrogenase (CAD), cinnamoyl CoA-reductase 1 (CCR1), 4-coumarate-CoA ligase (4CL), monolignol-lignin-specific glycosyltransferase, or aldehyde dehydrogenase (ALDH). In certain embodiments, the additional DNA sequence may comprise a mutated genomic copy of one or more lignin biosynthesis genes that disrupt expression of the gene or the function of the gene product.

In certain embodiments of the present disclosure, DNA constructs for plant transformation are provided. Vectors used for plant transformation may include, for example, plasmids, cosmids, YACs (yeast artificial chromosomes),BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. Utilization of cloning systems with large insert capacities will allow introduction of large DNA sequences comprising more than one selected gene. In accordance with the present disclosure, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. Expression cassettes which have been isolated from such vectors are useful for transformation. DNA segments used for transforming plant cells will generally comprise the cDNA, gene or genes which are desired to be introduced into and have expressed in the host cells. These DNA segments can, optionally, further include structures such as promoters, enhancers, polylinkers, and regulatory genes as desired. Exemplary promoters for expression of a nucleic acid sequence include plant promoter such as the CaMV 35S promoter, CaMV 19S, nos, Adh, sucrose synthase, alpha-tubulin, actin, cab, PEPCase or those associated with the R gene complex, maize ubiquitin promoter, and rice actin promoter. Tissue specific promoters and tissue specific enhancers are also contemplated to be useful, as are inducible promoters. The DNA sequence between the transcription initiation site and the start of the coding sequence, i.e., the untranslated leader sequence, can also influence gene expression. One may thus wish to employ a particular leader sequence with a transformation construct of the invention. Leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences are known to those of skill in the art in light of the present disclosure. Sequences that are derived from genes that are highly expressed in plants may be beneficial in particular embodiments.

Vectors for use in accordance with the present embodiments may be constructed to include an ocs enhancer element. This element was first identified as a 16 bp palindromic enhancer from the octopine synthase (ocs) gene of Agrobacterium, and is present in numerous other promoters. The use of an enhancer element, such as the ocs element and particularly multiple copies of the element, may act to increase the level of transcription from adjacent promoters when applied in the context of plant transformation. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue. Transformation constructs of the present disclosure may include a 3′ end DNA sequence that acts as a signal to terminate transcription and allow for the poly-adenylation of the mRNA produced by coding sequences operably linked to a promoter. It is envisioned that the native terminator of a transcription factor coding sequence may be used. Examples of terminators that are deemed to be useful in this context include those from the nopaline synthase gene of Agrobacterium tumefaciens (nos 3′ end), the terminator for the T7 transcript from the octopine synthase gene of Agrobacterium tumefaciens, and the 3′ end of the protease inhibitor I or II genes from potato or tomato. Regulatory elements such as an Adh intron, sucrose synthase intron or TMV omega element, may further be included where desired.

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequencesthat may not appear on the final polypeptide. Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Many examples of suitable marker proteins are known to the art and can be employed in the presently described embodiments. Many selectable marker coding regions are known including, but not limited to, neo, which provides kanamycin resistance and can be selected for using kanamycin, G418, paromomycin, bar, which confers bialaphos or phosphinothricin resistance; a mutant EPSPsynthase protein conferring glyphosate resistance; a nitrilase such as bxn from Klebsiella ozaenae which confers resistance to bromoxynil; a mutant acetolactate synthase (ALS) which confers resistance to imidazolinone, sulfonylurea or other ALS inhibiting chemicals, a methotrexate resistant DHFR, a dalapon dehalogenase that confers resistance to the herbicide dalapon; or a mutated anthranilate synthase thatconfers resistance to 5-methyl tryptophan.

Screenable markers that may be employed include a beta-glucuronidase (GUS) or uidA gene which encodes an enzyme for which various chromogenic substrates are known; an R-locus gene, which encodes a product that regulates the production of anthocyanin pigments (red color) in plant tissues; a beta-lactamase gene which encodes an enzyme for which various chromogenic substrates are known (e.g., PADAC, a chromogenic cephalosporin); a xy1E gene which encodes a catechol dioxygenase that can convert chromogenic catechols; an alpha-amylase gene; a tyrosinase gene which encodes an enzyme capable of oxidizing tyrosine to DOPA and dopaquinone which in turn condenses to form the easily-detectable compound melanin; a beta-galactosidase gene, which encodes an enzyme for which there are chromogenic substrates; a luciferase (lux) gene, which allows for bioluminescence detection; an aequorin gene which may be employed in calcium-sensitive bioluminescence detection; or a gene encoding for green fluorescent protein. The gene that encodes green fluorescent protein (GFP) is also contemplated as a particularly useful reporter gene. Expression of green fluorescent protein may be visualized in a cell or plant as fluorescence following illumination by particular wavelengths of light.

Constructs may be designed that are complementary to all or part of the promoter and other control regions, exons, introns or even exon-intron boundaries of a gene. Certain constructs may include regions complementary to intron/exon splice junctions. For example, one embodiment includes a construct with complementarity to regions within 50-200 bases of an intron-exon splice junction. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

In certain embodiments, portions of genomic DNA are combined with cDNA or synthetic sequences to generate specific constructs. For example, where an intron is desired in the ultimate construct, a genomic clone can be used. The cDNA or a synthesized polynucleotide may provide more convenient restriction sites for the remaining portion of the construct and, therefore, would be used for the rest of the sequence.

Suitable methods for genetic transformation of plant or other cells for use with the currently disclosed embodiments are believed to include virtually any method known in the art by which DNA can be introduced into a cell, such as by direct delivery of DNA, PEG-mediated transformation of protoplasts, desiccation/inhibition-mediated DNA uptake, electroporation, agitation with silicon carbide fibers; Agrobacterium-mediated transformation; and acceleration of DNA coated particles. Non-limiting examples of such techniques can be found in U.S. Pat. Nos. 5,302,523; 5,464,765; 5,591,616; 5,563,055; 5,550,318; 5,538,877; and 5,538,880, each of which is specifically incorporated herein by reference in its entirety. Through the application of techniques such as these, the cells of virtually any plant species, including biofuel crop species, may be stably transformed, and these cells developed into transgenic plants.

The term “crossing” as used herein refers to a technique of mating two genotypically-different plants that results in a transgene of a donor plant line being transferred into a receiving plant line initially having a different genotype. In one embodiment of the present disclosure, a transgene is introduced into a plant line by crossing a receiving (starting) plant line with a donor plant line that comprises a transgene as described herein. In one embodiment, this process could include the steps of (a) planting seeds of the receiving plant line and the donor plant line and grow them to a flower-bearing stage, (b) pollinating flowers from the receiving plant line with pollen from flowers of the donor plant line; and (c) harvesting seeds produced by the receiving plant line bearing the fertilized flowers.

The term “backcrossing” as used herein refers to a process including the steps of: (a) crossing a plant of a first genotype containing a desired gene, DNA sequence or element to a plant of a second genotype lacking the desired gene, DNA sequence or element forming progeny plants; (b) selecting one or more progeny plants containing the desired gene, DNA sequence or element; (c) crossing the progeny plant to a plant of the second genotype; and (d) repeating steps (b) and (c) for the purpose of transferring the desired gene, DNA sequence or element from a plant of the first genotype to a plant of the second genotype.

The term “introgression” as used herein refers to the process of backcross conversion by which a DNA element is introduced into a plant genotype. A plant genotype into which a DNA sequence has been introgressed may be referred to as a backcross converted genotype, line, inbred, or hybrid. Similarly, a plant genotype lacking the desired DNA sequence may be referred to as an unconverted genotype, line, inbred, or hybrid.

As noted above, antisense and interfering RNA treatments represent one way of altering lignin biosynthesis activity in accordance with the invention (e.g., by down-regulation of positive-regulating gene expression). In particular, constructs comprising a lignin biosynthesis coding sequence, including fragments thereof, in antisense orientation, or combinations of sense and antisense orientation, may be used to decrease or effectively eliminate the expression of a lignin biosynthesis gene in a plant and obtain an improvement in lignin profile as is described herein. Accordingly, this may be used to “knock-out” the function of a lignin biosynthesis coding sequence or homologous sequences thereof.

Techniques for using RNAi are well known in the art, for example it is known that double-stranded RNA is capable of directing the degradation of messenger RNA with sequence complementary to one or the other strand. Therefore, by expression of a particular coding sequence in sense and antisense orientation, either as a fragment or longer portion of the corresponding coding sequence, the expression of that coding sequence can be down-regulated.

Antisense, and in some aspects, RNAi methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation, or both, within a host cell, either in vitro or in vivo, such as within a host plant cell. In certain embodiments of the present disclosure, such an oligonucleotide may comprise any unique portion of a nucleic acid sequence provided herein. In certain embodiments of the present disclosure, such a sequence comprises at least 18, 30, 50, 75, or 100 or more contiguous nucleic acids of the nucleic acid sequence of gene related to lignin biosynthesis, and/or complements thereof, which may be in sense and/or antisense orientation. By including sequences in both sense and antisense orientation, increased suppression of the corresponding coding sequence may be achieved.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Sequences that are completely complementary have complete identity throughout their entire length and have no base mismatches. Other sequences with lower degrees of homology may also be used. For example, an RNAi or antisense construct that has limited regions of high homology, but also contains a non-homologous region could be designed. Methods for selection and design of sequences that generate RNAi are well known in the art.

Turning now to certain non-limiting embodiments of the present disclosure, various nucleic acid sequences and the protein sequences encoded thereby and described further below are shown in Table 1.

TABLE 1
Oryza sativa (rice) Transcription Factors

| SEQ ID NO: | Sequence Name | Sequence Type |
|---|---|---|
| 1 | OsSND2 | DNA |
| 2 | OsSND2 | Protein |
| 3 | WACH1 | DNA |
| 4 | WACH1 | Protein |
| 5 | OsMYB13a | DNA |
| 6 | OsMYB13a | Protein |
| 7 | OsMYB13b | DNA |
| 8 | OsMYB13b | Protein |
| 9 | WAHD1 | DNA |
| 10 | WAHD1 | Protein |
| 11 | WAP1 | DNA |
| 12 | WAP1 | Protein |
| 13 | WAHL1 | DNA |
| 14 | WAHL1 | Protein |
| 15 | OsMYB61a | DNA |
| 16 | OsMYB61a | Protein |
| 17 | OsMYB61b | DNA |
| 18 | OsMYB61b | Protein |

The rice (Oryza sativa) transcription factors OsSND2 and WACH1 have been identified as negative regulators of lignin synthesis, therefore increased expression of these negative regulators (or their orthologs from other species) via various inducible, native, or synthetic promoters can be used to reduce lignin synthesis. In particular, by over-expressing at least one (one or both) of the OsSND2 and WACH1 transcription factors, an accompanying decrease or reduction in lignin content in secondary cell walls of the transgenic plants can be achieved, as compared to a plant exhibiting normal expression of one or more of said transcription factors. The nucleic acid construct may comprise a heterologous promoter operably linked to the transcription factor nucleotide sequence that directs expression of the transcription factor in a plant cell. Other embodiments include using the transgenic plants having such modifications, including for example rice, for the production of biofuel feedstocks and livestock forage. For example, in certain embodiments, the present disclosure is directed to a plant, plant part, or plant cell having a nucleic acid sequence which encodes a protein having transcriptional repressor activity of at least one of OsSND2 and WACH1 transcriptional factors which comprise the sequence SEQ ID NO:1, or SEQ ID NO:3, respectively, or at least one homologous nucleic acid sequence which encodes a protein having transcriptional repressor activity of at least one of OsSND2 and WACH1 transcriptional factors and having at least 80% identity, or at least 85% identity, or at least 90% identity, or at least 91% identity, or at least 92% identity, or at least 93% identity, or at least 94% identity, or at least 95% identity, or at least 96% identity, or at least 97% identity, or at least 98% identity, or at least 99% identity to the sequence SEQ ID NO:1, or SEQ ID NO:3, respectively. Examples of such homologous nucleic acid sequences are shown in Tables A and B of the parent application, U.S. Provisional Application No. 62/872.990. each of which is included herein by reference its entirety.

The Oryza sativa transcription factors OsMYB 13a, OsMYB13b, WAHD1, WAHL1, and WAP1, OsMYB61a, or OsMYB61b have been identified herein as positive regulators. Decreased expression of these positive regulators (or their orthologs from other species, e.g. , see Tables C and D in U.S. Provisional Application No. 62/872,990 (each of which is included herein by reference its entirety). for examples of ortholog sequences of OsMYB 13a and OsMYB13b, respectively) can be used to decrease lignin synthesis in cases where decreased lignin content of the plant is desirable. Positive regulators of lignin synthesis can be targeted for reduced expression so as to reduce expression of cell wall modifying enzymes, precursors, or polymers that contribute to biomass recalcitrance. Reduced expression of positive regulators can be accomplished for example through RNAi technology, artificial-miRNA technology, antisense technology, and/or genome editing (such as by a CRISPR-Cas9/guide RNA system). In the case of RNA-mediated reduced expression, various promoters can be used to drive expression of the reducing molecule to accomplish cell-type or development-specific reduction of expression. Dominant-negative versions of the positive regulators might also be used to accomplish improved biomass properties. Genome editing can be used to alter motifs of specific promoter targets of these regulators.

As noted, in certain embodiments, plants according to the present disclosure comprise down-regulated expression of one or more genes which encode positive regulators of lignin synthesis, such as genes MYB 13a, MYB 13b, WAHD1, WAHL1, WAP1, MYB61a, or MYB61b, or their orthologs, wherein the lignin content of the plant is reduced when compared to a plant exhibiting normal expression of one or more of said genes. Down-regulation of one or more of the genes MYB13a, MYB13b, WAHD1, WAHL1, WAP1, MYB61a, or MYB61b, or their orthologs, may be accomplished by introducing a mutation that disrupts the gene by down-regulating expression of the gene, by abrogating expression entirely, or by rendering the gene product non-functional. For example, the mutation may be a point mutation, an insertion, a deletion, or any type of mutation known in the art that may result in down-regulation of a gene, and the mutation may be located in a coding or non-coding portion of the gene (e.g., in the promoter region). Mutations in the one or more genes can be accomplished by any of the methods well known to those in the art including random mutagenesis methods such as irradiation, random DNA integration (e.g., via a transposon), or by using a chemical mutagen. Moreover, in certain aspects, a gene may be mutated using a site-directed mutagenesis approach such as by using a genome editing procedure such as a CRISPR-Cas9/guideRNA system, homologous recombination vector, or by irradiation, T-DNA insertion, or chemical mutagenesis. These methods are known in the art, and one of skill will be able to identify such methods as appropriate in light of the present disclosure.

In a further embodiment, a selected DNA that causes down-regulation of expression of MYB13a, MYB13b, WAHD1, WAHL1, WAP1, MYB61a, or MYB61b, or their orthologs, may comprise a DNA molecule capable of expressing a nucleic acid sequence complementary to all or a portion of said gene sequence or a messenger RNA (mRNA) transcribed from said sequence. Thus, in some aspects, a transgenic plant may comprise a nucleic acid construct an antisense, RNAi, siRNA, shRNA, guideRNA, or miRNA sequence for down-regulation of one or more of the MYB13a, MYB13b, WAHD1, WAHL1, WAP1, MYB61a, or MYB61b genes, or their orthologs. In certain embodiments, a plant according to the present disclosure may comprise an RNAi construct comprising all or a portion of SEQ ID NOS: 5, 7, 9, 11, 13, 15, and 17 or a complementary sequence thereof. Such a construct may be engineered to target some or a portion of the MYB13a, MYB13b, WAHD1, WAHL1, WAP1, MYB61a, or MYB61b genes to achieve down-regulation of said genes. In some embodiments, such a construct may be engineered to target introns or exons, or both, of a particular gene. For example, a transgenic plant may comprise a promoter that expresses a sequence complimentary to all or a portion of the nucleic acid sequence MYB13a, MYB13b, WAHD1, WAHL1, WAP1, MYB61a, or MYB61b from the plant. The promoter sequence may be selected from the group consisting of developmentally-regulated, cell-specific, organelle-specific, tissue-specific, xylem-specific, leaf-specific, root-specific, inducible, and constitutive promoters, and combinations thereof. Examples of orthologs of WAP1, WAHL1, and WAHD1 sequences from other species are shown in Tables E, F and G in U.S. Provisional Application No. 62/872,990 (each of which is included herein by reference its entirety).

As noted, the rice transcription factors OsMYB13a, OsMYB13b, WAHD1, WAHL1, WAP1, OsMYB61a, and OsMYB61b have been identified herein as positive regulators of lignin synthesis, therefore increased expression of these positive regulators (or their orthologs from other species) via various inducible, native, or synthetic promoters can be used to increase lignin synthesis where that is desirable. In particular, by over-expressing at least one of the OsMYB13a, OsMYB13b, WAHD1, WAHL1, WAP1, OsMYB61a, and OsMYB61b transcription factors, an accompanying increase in lignin content in secondary cell walls of the transgenic plants can be achieved, as compared to a plant exhibiting normal expression of one or more of said transcription factors. The nucleic acid construct may comprise a heterologous promoter operably linked to the transcription factor nucleotide sequence that directs expression of the transcription factor in a plant cell. The promoter sequence may be selected from the group consisting of developmentally-regulated, cell-specific, organelle- specific, tissue-specific, xylem-specific, leaf-specific, root-specific, inducible, and constitutive promoters, and combinations thereof. Other embodiments include using the transgenic plants having such modifications, including for example rice, for the production of biofuel feedstocks and livestock forage. For example, in certain embodiments, the present disclosure is directed to a plant, plant part, or plant cell having a nucleic acid sequence which encodes a protein having up-regulating activity of at least one of OsMYB13a, OsMYB13b, WAHD1, WAHL1, WAP1, OsMYB61a, and OsMYB61b transcriptional factors, wherein the nucleic acid sequence has a sequence selected from SEQ ID NOS: 5, 7, 9, 11, 13, 15 and 17, respectively, or wherein the nucleic acid sequence has at least 80% identity, or at least 85% identity, or at least 90% identity, or at least 91% identity, or at least 92% identity, or at least 93% identity, or at least 94% identity, or at least 95% identity, or at least 96% identity, or at least 97% identity, or at least 98% identity, or at least 99% identity to the sequences SEQ ID NOS: 5, 7, 9, 11, 13, 15 and 17, respectively, or at least one homologous nucleic acid sequence which encodes a protein having up-regulating activity of at least one of OsMYB13a, OsMYB13b, WAHD1, WAHL1, WAP1, OsMYB61a, and OsMYB61b transcriptional factors, wherein the protein has at least 80% identity, or at least 85% identity, or at least 90% identity, or at least 91% identity, or at least 92% identity, or at least 93% identity, or at least 94% identity, or at least 95% identity, or at least 96% identity, or at least 97% identity, or at least 98% identity, or at least 99% identity to the sequences SEQ ID NOS: 6, 8, 10, 12, 14, 16, and 18, respectively.

The present disclosure is further described in examples shown in Appendix 1, Appendix 2, and Appendix 3 of U.S. Provisional Application No. 62/872,990 (each of which is included herein by reference its entirety). These examples are provided for purposes of illustration only and are not intended to be limiting unless otherwise specified. Thus, the present disclosure should in no way be construed as being limited to the examples, but rather, should be construed to encompass any and all variations which become evident as a result of the teachings provided herein.

In at least certain embodiments, the present disclosure is directed to the plants, plant parts, cells, compositions, and methods described in of the following non-limiting clauses.
Clause 1. A transgenic plant, plant part, or plant cell, comprising a recombinant DNA construct having a nucleic acid sequence which encodes a protein having transcriptional repressor activity of at least one of OsSND2 and WACH1 transcriptional factors, wherein the nucleic acid sequence is operably linked to a heterologous promoter sequence which increases expression of said at least one nucleic acid sequence as compared to a DNA construct absent said heterologous promoter sequence, and wherein the recombinant DNA construct reduces content of at least one of lignin, hemicellulose, and cellulose in the transgenic plant, plant part, or plant cell as compared to a non-transgenic control plant, plant part, or plant cell of the same species lacking said recombinant DNA construct.
Clause 2. The transgenic plant, plant part, or plant cell of clause 1, wherein the nucleic acid sequence which encodes a protein having transcriptional repressor activity of at least one of OsSND2 and WACH1 transcriptional factors comprises at least one of:
   (a) a nucleic acid comprising the sequence of at least one of SEQ ID NO:1 and SEQ ID NO:3;
   (b) a nucleic acid sequence exhibiting at least 80% sequence identity to the nucleic acid sequence of at least one of SEQ ID NO:1 and, SEQ ID NO:3;
   (c) a nucleic acid sequence that encodes a polypeptide having an amino acid sequence of at least one of SEQ ID NO:2 and SEQ ID NO:4; and
   (d) a nucleic acid sequence that encodes a polypeptide that is at least 80% identical to an amino acid sequence of at least one of SEQ ID NO:2 and SEQ ID NO:4.
Clause 3. The plant, plant part, or plant cell of clause 1, selected from the group consisting of switchgrass (Panicum virgatum), giant reed (Arundo donax), reed canarygrass (Phalaris arundinacea), MiscanthusXgiganteus, Miscanthus sp., Sericea lespedeza, corn, sugarcane, sorghum, millet, ryegrass, rye, timothy grass, Kochia (Kochia scoparia), soybean, alfalfa, clover, sunn hemp, kenaf, bahiagrass, bermudagrass, dallisgrass, pangolagrass, big bluestem, little bluestem, indiangrass, fescue, centipede grass (Eremochloa ophiuroides), Dactylis sp., Brachypodium distachyon, smooth bromegrass, orchardgrass, Kentucky bluegrass, poplar, rice, cotton, red sage, apple, Vitis vinifera, castor bean (Ricinus communis), hops (Humulus lupulus), Dahlia, orchid sp., mustards (e.g., Brassica rapa), kudzu (Pueraria lobata), wheat, eucalyptus, alder, and cedar.
Clause 4. The transgenic plant, plant part, or plant cell of clause 1, wherein the plant is rice.
Clause 5. The transgenic plant, plant part, or plant cell of clause 1, further defined as an R0 transgenic plant.
Clause 6. The transgenic plant, plant part, or plant cell of clause 1, further defined as a progeny plant of any generation of an R0 transgenic plant, wherein the transgenic plant has inherited the recombinant DNA construct.
Clause 7. The transgenic plant, plant part, or plant cell of clause 1, wherein the heterologous promoter sequence is selected from the group consisting of cell-specific, organelle-specific, tissue-specific, xylem-specific, leaf-specific, root-specific, inducible, and constitutive, and combinations thereof.
Clause 8. A recombinant DNA construct comprising at least one of:
   (a) a nucleic acid comprising the sequence of at least one of SEQ ID NO:1 and SEQ ID NO:3;
   (b) a nucleic acid sequence exhibiting at least 80% sequence identity to the nucleic acid sequence of at least one of SEQ ID NO:1 and, SEQ ID NO:3;
   (c) a nucleic acid sequence that encodes a polypeptide having an amino acid sequence of at least one of SEQ ID NO:2 and SEQ ID NO:4; and
   (d) a nucleic acid sequence that encodes a polypeptide that is at least 80% identical to an amino acid sequence of at least one of SEQ ID NO:2 and SEQ ID NO:4;
   and wherein the nucleic acid sequence is operably linked to a heterologous promoter sequence which increases expression of said at least one nucleic acid sequence as compared to a DNA construct absent said heterologous promoter sequence, wherein the nucleic acid sequence encodes a protein having transcriptional repressor activity of at least one of OsSND2 and WACH1 transcriptional factors, and wherein introduction of the recombinant DNA construct in a transgenic plant reduces content of at least one of lignin, hemicellulose, and cellulose in the transgenic plant as compared to a non-transgenic control plant of the same species lacking said recombinant DNA construct.
Clause 9. The recombinant DNA construct of clause 8, wherein the heterologous promoter sequence is selected from the group consisting of cell-specific, organelle-specific, tissue-specific, xylem-specific, leaf-specific, root-specific, inducible, and constitutive, and combinations thereof.
Clause 10. A transgenic plant cell, plant part, or plant comprising the recombinant DNA construct of clause 8.
Clause 11. A method of modifying the secondary cell wall of a plant, comprising: introducing into the plant the recombinant DNA construct of clause 8, wherein expression of the at least one of OsSND2 and WACH1 transcriptional factors is overexpressed, thereby reducing biosynthesis of at least one of lignin, hemicellulose, and cellulose in the secondary cell walls of the plant.
Clause 12. The method of clause 10, wherein the secondary cell wall comprises an increased content of fermentable carbohydrates.
Clause 13. A method for producing biomass, feedstock, forage, feed, or silage, comprising obtaining the transgenic plant or plant part of clause 1, and harvesting feedstock, biomass, forage, feed, or silage therefrom.
Clause 14. The method of clause 13, further comprising producing a biofuel from said biomass, feedstock, forage, feed, or silage.
Clause 15. A transgenic plant, plant part, or plant cell exhibiting artificially down-regulated expression of at least one of genes MYB13a, MYB13b, WAHD1, WAHL1, and WAP1, wherein the plant comprises a recombinant nucleic acid construct directed against the at least one of said genes and exhibits reduced content of at least one of lignin, hemicellulose, and cellulose.
Clause 16. The transgenic plant, plant part, or plant cell of clause 15, wherein the genes MYB13a, MYB13b, WAHD1, WAHL1, and WAP1 comprise the sequences SEQ ID NO:5, SEQ ID NO:7, SEQ ID NO:9, SEQ ID NO:11, and SEQ ID NO: 13, respectively, or nucleic acid sequences having at least 80% identity thereto, respectively.
Clause 17. The transgenic plant, plant part, or plant cell of clause 15, further exhibiting artificially down-regulated expression of at least one of genes MYB61a and MYB61b.
Clause 18. The transgenic plant, plant part, or plant cell of clause 17, wherein the genes MYB61a and MYB61b comprise the sequences SEQ ID NO: 15 and SEQ ID NO: 17, respectively, or nucleic acid sequences having at least 80% identity thereto, respectively.
Clause 19. The transgenic plant, plant part, or plant cell of clause 15, wherein the recombinant nucleic acid is an RNAi construct.
Clause 20. The transgenic plant, plant part, or plant cell of clause 15, wherein the recombinant nucleic acid construct comprises all or a portion of a nucleic acid sequence selected from SEQ ID NO:5, SEQ ID NO:7, SEQ ID NO:9, SEQ ID NO:11, and SEQ ID NO: 13, and complementary sequences thereof.
Clause 21. The transgenic plant, plant part, or plant cell of clause 15, wherein the plant is a forage plant, a biofuel crop, a cereal crop, or an industrial plant.
Clause 22. The transgenic plant, plant part, or plant cell of clause 15, wherein the plant part is a protoplast, cell, meristem, root, pistil, anther, flower, seed, embryo, stalk, or petiole.

0Clause 23. A method of modifying the secondary cell wall of a plant, comprising: introducing into the plant the recombinant nucleic acid construct of clause 15, wherein expression of the at least one of MYB13a, MYB13b, WAHD1, WAHL1, and WAP1 transcriptional factors is underexpressed, thereby reducing biosynthesis of at least one of lignin, hemicellulose, and cellulose in the secondary cell walls of the plant.
Clause 24. The method of clause 23, wherein the recombinant nucleic acid construct further comprises underexpression of at least one of MYB61a and MYB61b transcriptional factors
Clause 25. The method of clause 23, wherein the secondary cell wall comprises an increased content of fermentable carbohydrates.
Clause 26. A method for producing biomass, feedstock, forage, feed, or silage, comprising obtaining the transgenic plant, or plant part of clause 15, and harvesting feedstock, biomass, forage, feed, or silage therefrom.
Clause 27. The method of clause 26, further comprising producing a biofuel from said biomass, feedstock, forage, feed, or silage.
Clause 28. A transgenic plant, plant part, or plant cell, comprising a recombinant DNA construct having a nucleic acid sequence which encodes a protein having positive regulator activity of at least one of MYB13a, MYB13b, WAHD1, WAHL1, and WAP1 transcriptional factors, wherein the nucleic acid sequence is operably linked to a heterologous promoter sequence which increases expression of said at least one nucleic acid sequence as compared to a DNA construct absent said heterologous promoter sequence, and wherein the recombinant DNA construct increases content of at least one of lignin, hemicellulose, and cellulose in the transgenic plant, plant part, or plant cell as compared to a non-transgenic control plant, plant part, or plant cell of the same species lacking said recombinant DNA construct.
Clause 29. The transgenic plant, plant part, or plant cell of clause 28, wherein the nucleic acid sequence which encodes a protein having positive regulator activity of at least one of MYB13a, MYB 13b, WAHD1, WAHL1, and WAP1 transcriptional factors comprises the sequence SEQ ID NO:5, SEQ ID NO:7, SEQ ID NO:9, SEQ ID NO:11, and SEQ ID NO:13, respectively, or nucleic acid sequences having at least 80% identity thereto.
Clause 30. The transgenic plant, plant part, or plant cell of clause 28, further exhibiting artificially up-regulated expression of at least one of genes MYB61a and MYB61b.
Clause 31. The transgenic plant, plant part, or plant cell of clause 30, wherein the genes MYB61a and MYB61b comprise the sequences SEQ ID NO: 15 and SEQ ID NO: 17, respectively, or nucleic acid sequences having at least 80% identity thereto, respectively.
Clause 32. The plant, plant part, or plant cell of clause 28, selected from the group consisting of switchgrass (Panicum virgatum), giant reed (Arundo donax), reed canarygrass (Phalaris arundinacea), MiscanthusXgiganteus, Miscanthus sp., Sericea lespedeza, corn, sugarcane, sorghum, millet, ryegrass, rye, timothy grass, Kochia (Kochia scoparia), soybean, alfalfa, clover, sunn hemp, kenaf, bahiagrass, bermudagrass, dallisgrass, pangolagrass, big bluestem, little bluestem, indiangrass, fescue, centipede grass (Eremochloa ophiuroides), Dactylis sp., Brachypodium distachyon, smooth bromegrass, orchardgrass, Kentucky bluegrass, poplar, rice, cotton, red sage, apple, Vitis vinifera, castor bean (Ricinus communis), hops (Humulus lupulus), Dahlia, orchid sp., mustards (e.g., Brassica rapa), kudzu (Pueraria lobata), wheat, eucalyptus, alder, and cedar.
Clause 33. The transgenic plant, plant part, or plant cell of clause 28, wherein the plant is rice.
Clause 34. The transgenic plant, plant part, or plant cell of clause 28, further defined as an R0 transgenic plant.
Clause 35. The transgenic plant, plant part, or plant cell of clause 28, further defined as a progeny plant of any generation of an RO transgenic plant, wherein the transgenic plant has inherited the recombinant DNA construct.
Clause 36. A recombinant DNA construct comprising at least one of:
   (a) a nucleic acid comprising the sequence of at least one of SEQ ID NO:5, SEQ ID NO:7, SEQ ID NO:9, SEQ ID NO:11, and SEQ ID NO:13;
   (b) a nucleic acid sequence exhibiting at least 80% sequence identity to the nucleic acid sequence of at least one of SEQ ID NO:5, SEQ ID NO:7, SEQ ID NO:9, SEQ ID NO:11, and SEQ ID NO:13;
   (c) a nucleic acid sequence that encodes a polypeptide having an amino acid sequence of at least one of SEQ ID NO:6, SEQ ID NO:8, SEQ ID NO:10, SEQ ID NO:12, and SEQ ID NO:14; and
   (d) a nucleic acid sequence that encodes a polypeptide that is at least 80% identical to an amino acid sequence of at least one of SEQ ID NO:6, SEQ ID NO:8, SEQ ID NO:10, SEQ ID NO:12, and SEQ ID NO:14;
   and wherein the nucleic acid sequence is operably linked to a heterologous promoter sequence which increases expression of said at least one nucleic acid sequence as compared to a DNA construct absent said heterologous promoter sequence, wherein the nucleic acid sequence encodes a protein having positive regulator activity of at least one of MYB13a, MYB13b, WAHD1, WAHL1, and WAP1 transcriptional factors, and wherein introduction of the recombinant DNA construct in a transgenic plant increases content of at least one of lignin, hemicellulose, and cellulose in the transgenic plant as compared to a non-transgenic control plant of the same species lacking said recombinant DNA construct.
Clause 37. The recombinant DNA construct of clause 36, further comprising at least one of the nucleic acid sequences SEQ ID NO: 15 and SEQ ID NO: 17, respectively, or nucleic acid sequences having at least 80% identity thereto, respectively, wherein the nucleic acid sequence encodes a protein having positive regulator activity of at least one of MYB61a and MYB61b.
Clause 38. The recombinant DNA construct of clause 36, wherein the heterologous promoter sequence is selected from the group consisting of cell-specific, organelle-specific, tissue-specific, xylem-specific, leaf-specific, root-specific, inducible, and constitutive, and combinations thereof.
Clause 39. A transgenic plant cell, plant part, or plant comprising the recombinant DNA construct of clause 36.
Clause 40. A method of modifying the secondary cell wall of a plant, comprising: introducing into the plant the recombinant DNA construct of clause 36, wherein expression of the at least one of MYB13a, MYB13b, WAHD1, WAHL1, and WAP1 transcriptional factors is overexpressed, thereby increasing content of at least one of lignin, hemicellulose, and cellulose in the secondary cell walls of the plant.
Clause 41. A method for producing biomass, feedstock, forage, feed, or silage, comprising obtaining the transgenic plant or plant part of clause 39, and harvesting feedstock, biomass, forage, feed, or silage therefrom.
Clause 42. The method of clause 41, further comprising producing a biofuel from said biomass, feedstock, forage, feed, or silage.
Clause 43. A transgenic plant, plant part, or plant cell exhibiting artificially down-regulated expression of at least one of genes OsSND2 and WACH1, wherein the plant comprises a recombinant nucleic acid construct directed against the at least one of said genes and exhibits increased content of at least one of lignin, hemicellulose, and cellulose.
Clause 44. The transgenic plant, plant part, or plant cell of clause 43, wherein the genes OsSND2 and WACH1 comprise the sequences SEQ ID NO:1, and SEQ ID NO:3, respectively, or nucleic acid sequences having at least 80% identity thereto, respectively.
Clause 45. The transgenic plant, plant part, or plant cell of clause 43, wherein the recombinant nucleic acid is an RNAi construct.
Clause 46. The transgenic plant, plant part, or plant cell of clause 43, wherein the recombinant nucleic acid construct comprises all or a portion of a nucleic acid sequence selected from SEQ ID NO:1 and SEQ ID NO:3, and complementary sequences thereof.
Clause 47. The transgenic plant, plant part, or plant cell of clause 43, wherein the plant is a forage plant, a biofuel crop, a cereal crop, or an industrial plant.
Clause 48. The transgenic plant, plant part, or plant cell of clause 43, wherein the plant part is a protoplast, cell, meristem, root, pistil, anther, flower, seed, embryo, stalk, or petiole.
Clause 49. A method of modifying the secondary cell wall of a plant, comprising: introducing into the plant the recombinant nucleic acid construct of clause 43, wherein expression of the at least one of OsSND2 and WACH1 transcriptional factors is underexpressed, thereby increasing biosynthesis of at least one of lignin, hemicellulose, and/or cellulose in the secondary cell walls of the plant.
Clause 50. A method for producing biomass, feedstock, forage, feed, or silage, comprising obtaining the transgenic plant, or plant part of clause 43, and harvesting feedstock, biomass, forage, feed, or silage therefrom.
Clause 51. The method of clause 50, further comprising producing a biofuel from said biomass, feedstock, forage, feed, or silage.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

What is claimed is:

1. A transgenic plant, plant part, or plant cell, comprising a recombinant DNA construct having a nucleic acid sequence which encodes a protein having transcriptional repressor activity of at least one of OsSND2 and WACH1 transcriptional factors, wherein the nucleic acid sequence is operably linked to a heterologous promoter sequence which increases expression of said at least one nucleic acid sequence as compared to a DNA construct absent said heterologous promoter sequence, and wherein the recombinant DNA construct reduces content of at least one of lignin, hemicellulose, and cellulose in the transgenic plant, plant part, or plant cell as compared to a non-transgenic control plant, plant part, or plant cell of the same species lacking said recombinant DNA construct.

2. The transgenic plant, plant part, or plant cell of claim 1, wherein the nucleic acid sequence which encodes a protein having transcriptional repressor activity of at least one of OsSND2 and WACH1 transcriptional factors comprises at least one of:
(a) a nucleic acid comprising the sequence of at least one of SEQ ID NO:1 and SEQ ID NO:3;
(b) a nucleic acid sequence exhibiting at least 80% sequence identity to the nucleic acid sequence of at least one of SEQ ID NO:1 and, SEQ ID NO:3;
(c) a nucleic acid sequence that encodes a polypeptide having an amino acid sequence of at least one of SEQ ID NO:2 and SEQ ID NO:4; and
(d) a nucleic acid sequence that encodes a polypeptide that is at least 80% identical to an amino acid sequence of at least one of SEQ ID NO:2 and SEQ ID NO:4.

3. The plant, plant part, or plant cell of claim 1, selected from the group consisting of switchgrass (Panicum virgatum), giant reed (Arundo donax), reed canarygrass (Phalaris arundinacea), MiscanthusXgiganteus, Miscanthus sp., Sericea lespedeza, corn, sugarcane, sorghum, millet, ryegrass, rye, timothy grass, Kochia (Kochia scoparia), soybean, alfalfa, clover, sunn hemp, kenaf, bahiagrass, bermudagrass, dallisgrass, pangolagrass, big bluestem, little bluestem, indiangrass, fescue, centipede grass (Eremochloa ophiuroides), Dactylis sp., Brachypodium distachyon, smooth bromegrass, orchardgrass, Kentucky bluegrass, poplar, rice, cotton, red sage, apple, Vitis vinifera, castor bean (Ricinus communis), hops (Humulus lupulus), Dahlia, orchid sp., mustards (e.g., Brassica rapa), kudzu (Pueraria lobata), wheat, eucalyptus, alder, and cedar.

4. The transgenic plant, plant part, or plant cell of claim 1, wherein the plant is rice.

5. The transgenic plant, plant part, or plant cell of claim 1, wherein the plant part is a protoplast, cell, meristem, root, pistil, anther, flower, seed, embryo, stalk, or petiole.

6. The transgenic plant, plant part, or plant cell of claim 1, further defined as an R0 transgenic plant.

7. The transgenic plant, plant part, or plant cell of claim 1, further defined as a progeny plant of any generation of an R0 transgenic plant, wherein the transgenic plant has inherited the recombinant DNA construct.

8. The transgenic plant, plant part, or plant cell of claim 1, wherein the heterologous promoter sequence is selected from the group consisting of cell-specific, organelle-specific, tissue-specific, xylem-specific, leaf-specific, root-specific, inducible, and constitutive, and combinations thereof.

9. A method for producing biomass, feedstock, forage, feed, or silage, comprising obtaining the transgenic plant, plant part, or plant cell of claim 1, and harvesting feedstock, biomass, forage, feed, or silage therefrom.

10. The method of claim 9, wherein the transgenic plant, plant part, or plant cell has an increased content of fermentable carbohydrates.

11. The method of claim 9, further comprising producing a biofuel from said biomass, feedstock, forage, feed, or silage.

12. A transgenic plant, plant part, or plant cell exhibiting artificially down-regulated expression of at least one of genes MYB13a, MYB13b, WAHD1, WAHL1, and WAP1, wherein the plant comprises a recombinant nucleic acid construct directed against the at least one of said genes and exhibits reduced content of at least one of lignin, hemicellulose, and cellulose.

13. The transgenic plant, plant part, or plant cell of claim 12, wherein the genes MYB13a, MYB 13b, WAHD1, WAHL1, and WAP1 comprise the sequences SEQ ID NO:5, SEQ ID NO:7, SEQ ID NO:9, SEQ ID NO:11, and SEQ ID NO: 13, respectively, or nucleic acid sequences having at least 80% identity thereto, respectively.

14. The transgenic plant, plant part, or plant cell of claim 12, further exhibiting artificially down-regulated expression of at least one of genes MYB61a and MYB61b.

15. The transgenic plant, plant part, or plant cell of claim 14, wherein the genes MYB61a and MYB61b comprise the sequences SEQ ID NO: 15 and SEQ ID NO: 17, respectively, or nucleic acid sequences having at least 80% identity thereto, respectively.

16. The transgenic plant, plant part, or plant cell of claim 12, wherein the recombinant nucleic acid is an RNAi construct.

17. The transgenic plant, plant part, or plant cell of claim 12, wherein the recombinant nucleic acid construct comprises all or a portion of a nucleic acid sequence selected from SEQ ID NO:5, SEQ ID NO:7, SEQ ID NO:9, SEQ ID NO:11, and SEQ ID NO: 13, and complementary sequences thereof.

18. The transgenic plant, plant part, or plant cell of claim 12, wherein the plant is a forage plant, a biofuel crop, a cereal crop, or an industrialplant.

19. The plant, plant part, or plant cell of claim 12, selected from the group consisting of switchgrass (Panicum virgatum), giant reed (Arundo donax), reed canarygrass (Phalaris arundinacea), MiscanthusXgiganteus, Miscanthus sp., Sericea lespedeza, corn, sugarcane, sorghum, millet, ryegrass, rye, timothy grass, Kochia (Kochia scoparia), soybean, alfalfa, clover, sunn hemp, kenaf, bahiagrass, bermudagrass, dallisgrass, pangolagrass, big bluestem, little bluestem, indiangrass, fescue, centipede grass (Eremochloa ophiuroides), Dactylis sp., Brachypodium distachyon, smooth bromegrass, orchardgrass, Kentucky bluegrass, poplar, rice, cotton, red sage, apple, Vitis vinifera, castor bean (Ricinus communis), hops (Humulus lupulus), Dahlia, orchid sp., mustards (e.g., Brassica rapa), kudzu (Pueraria lobata), wheat, eucalyptus, alder, and cedar.

20. The transgenic plant, plant part, or plant cell of claim 12, wherein the plant part is a protoplast, cell, meristem, root, pistil, anther, flower, seed, embryo, stalk, or petiole.


SEQUENCE LISTING (18 sequences)

SEQ ID NO: 1 - DNA, 1008 bp, Oryza sativa
atgacatggtgcaacagcttcaacgacgtgcgcgccgtggagaacaacctggccaccgccgccgccgtggccgcggccaagaagcagcagcagcagcagcaggtctcccagcacgtcaacctcatcaagacctgcccctcctgcggccaccgcgcgcagtatgagcagttgcaggcagcggcggcggcgacgatccaggacctgccggggctgccggccggggtgaagttcgacccgacggaccaggagctgctggagcacctggaagggaaggcgaggcctgacgcgcggaagctccaccccctcatcgacgagttcatccccaccatcgagggcgagaacggcatctgctacacccatcccgagaggcttccaggagtcggcaaggacggcctgatccggcacttcttccaccgcccgtcgaaggcgtacacgacggggacgcggaagcggcggaaggtgcacaccgacgagcagggcggcgagacgcggtggcacaagaccggcaagacgcggccggtgttcaccggcggcaagctgaaggggtacaagaagatcctggtgctctacaccaactacggcaagcagcgcaagccggagaagaccaactgggtgatgcaccagtaccacctgggctccgacgaggaggagaaggacggcgagctcgtcgtgtccaaggtgttctaccagacgcagccgaggcagtgcggcggcggctcggcagcgacggccaaggacctctccgtcgacctcgtcgccggtaacaacatcaaggctagcaatgctgctgctgaacaccaccataacgacggcgtcggcggcggcggccacggtggcaacaacagcagcatgctgaaggaagctgcaggcattgtagatttctacaacccagcggcggcgctaatcggctacagccaggcggctcctaacaacagggcggctgcttctgctcatttgacgatgcctaactttgaggtgcacacaggaggtgcaggctttggcccctga

SEQ ID NO: 2 - protein, 335 aa, Oryza sativa
MTWCNSFNDVRAVENNLATAAAVAAAKKQQQQQQVSQHVNLIKTCPSCGHRAQYEQLQAAAAATIQDLPGLPAGVKFDPTDQELLEHLEGKARPDARKLHPLIDEFIPTIEGENGICYTHPERLPGVGKDGLIRHFFHRPSKAYTTGTRKRRKVHTDEQGGETRWHKTGKTRPVFTGGKLKGYKKILVLYTNYGKQRKPEKTNWVMHQYHLGSDEEEKDGELVVSKVFYQTQPRQCGGGSAATAKDLSVDLVAGNNIKASNAAAEHHHNDGVGGGGHGGNNSSMLKEAAGIVDFYNPAAALIGYSQAAPNNRAAASAHLTMPNFEVHTGGAGFGP

SEQ ID NO: 3 - DNA, 1029 bp, Oryza sativa
atgggcgtccaggaggaggccgcgctcgccgccgccgtcgtcaagggcaagcggagcaagcgccagcgggcgcacgcggcggtggtcccgatcgccgcgtcggcgacggcggaggaggaggagtccatggagtcgtcgctgtcgctgtcgggcggcgccgcggcggaggggtcgtcgacgacgtcgccgttgttgacgacgacggccgcacggggcgacgaggccgtgtccgggtgcgtcaccgaggaggaggaggacatggcgctctgcctcatgctgctcgccagcggcggccacggcgagcgcgcgccggacgccgaggcggtggcggcgaaggaggccaagttccggagccggcggccggcggacggcgccggcgccggggagttcgtgtacgagtgcaagacgtgcagcaagtgcttcccgtcgttccaggcgctcggcggccaccgcaccagccacaagaagcctcgcctggtcgcgccgccggcggccatggaaccggcagccgatgacaagatcaagccgaccatcccggagacagccgccgccgccgccgaggagaagccgcccaagccatcgccgccacgaccaccggcctcgaggccgatcgccaccgatccaacagtgctcgcgatcccggtgatcccgaagcaagaagtgctcgacgccaccaactccgccgccattgccagcgtctccaagcagccacgtgtccacgagtgctccatctgcggcgccgagttcgcctccggccaggcgctcggcggccacatgcggcggcaccgcccgctcataccggcgtcggcgtcgtccgcggtggtgtccgtgctcgacgccgtcgacgcgccgaggcagaaggagaagagcctcctcgagctggatctcaacatgccggcgccctgcgacgacgcgaccgccgagacgacgacgacgtcgtccgccgccacctcgccggcgttcgccttcgccgtcagcgacaggtcgccgctgctggtgccggcggcgctggtcggctgccattactag

SEQ ID NO: 4 - protein, 342 aa, Oryza sativa
MGVQEEAALAAAVVKGKRSKRQRAHAAVVPIAASATAEEEESMESSLSLSGGAAAEGSSTTSPLLTTTAARGDEAVSGCVTEEEEDMALCLMLLASGGHGERAPDAEAVAAKEAKFRSRRPADGAGAGEFVYECKTCSKCFPSFQALGGHRTSHKKPRLVAPPAAMEPAADDKIKPTIPETAAAAAEEKPPKPSPPRPPASRPIATDPTVLAIPVIPKQEVLDATNSAAIASVSKQPRVHECSICGAEFASGQALGGHMRRHRPLIPASASSAVVSVLDAVDAPRQKEKSLLELDLNMPAPCDDATAETTTTSSAATSPAFAFAVSDRSPLLVPAALVGCHY

SEQ ID NO: 5 - DNA, 777 bp, Oryza sativa
atggggagggcgccgtgctgcgagaagatggggctgaagagggggccgtggacggcggaggaggacaggatcctggtggcgcacatcgagcggcacgggcacagcaactggcgcgcgctgccgaggcaggccggccttctccgctgcggcaagagctgccgcctccggtggatcaactacctccgccccgacatcaagcgcggcaacttcacccgcgaggaggaggacgccatcatccacctccacgaccttctcggcaaccgatggtccgcgattgcagcgaggctgccggggaggacggacaacgagatcaagaatgtgtggcacactcacctcaagaagcggctggagccgaagccgtcgtccggccgggaagccgccgcgcccaagcgaaaggcgaccaagaaggctgcggcggtggcggtggcgatcgacgttccgaccaccgtgccggtgtcgccggagcagtcgctctcgaccacgacgacgtcggccgccaccaccgaggagtactcgtactcgatggcctcctccgcggatcacaacaccacggacagtttcacctcggaggaggagttccagatcgacgacagcttctggtcggagacgctggcaatgacggtggacagcaccgactccgggatggagatgagcggcggcgatcctctcggcgcgggcggtgcctcgccgtcgtcgagcaacgacgacgacatggacgacttctggctcaagctgttcatccaggccggtggcatgcagaatttgccccagatttaa

SEQ ID NO: 6 - protein, 258 aa, Oryza sativa
MGRAPCCEKMGLKRGPWTAEEDRILVAHIERHGHSNWRALPRQAGLLRCGKSCRLRWINYLRPDIKRGNFTREEEDAIIHLHDLLGNRWSAIAARLPGRTDNEIKNVWHTHLKKRLEPKPSSGREAAAPKRKATKKAAAVAVAIDVPTTVPVSPEQSLSTTTTSAATTEEYSYSMASSADHNTTDSFTSEEEFQIDDSFWSETLAMTVDSTDSGMEMSGGDPLGAGGASPSSSNDDDMDDFWLKLFIQAGGMQNLPQI

SEQ ID NO: 7 - DNA, 774 bp, Oryza sativa
atggggagggctccgtgctgcgagaagatggggctcaagaagggtccatggacgccggaggaggacaaggtcctcgtcgcccacatccagcgccacggccacggcaactggcgcgccctgcccaagcaagccgggctgctgcgttgcggcaagagctgccggctccggtggatcaactacctgcggccggacatcaagcggggcaacttctccaaggaggaggaggacaccatcatccatctccacgagctgcttggcaacaggtggtccgcaattgccgccaggttgcccgggaggacggacaacgagatcaagaacgtgtggcacacccacctcaagaagcgcctcgatgcgccggctcagggcggtcatgtcgcggcgagcggcggcaagaagcacaagaagccgaagagcgcgaagaagccagccgccgccgccgccgcgccgccggcgtcgcccgagcggtccgcctcgtcgtcggtgacggagtcctcgatggcctcgtcggtggcggaggagcacggcaacgccgggatcagctcggcgtccgcgtccgtgtgcgccaaggaggagagctccttcacctcggcttccgaggagttccagatcgacgacagcttctggtcggagacgctgtcgatgccgctggacgggtacgacgtgtccatggagcccggcgacgcgttcgtcgcgccgccatccgccgacgacatggactactggctcggagtgttcatggagtccggcgaagcgcaagacttgccgcagatctag

SEQ ID NO: 8 - protein, 257 aa, Oryza sativa
MGRAPCCEKMGLKKGPWTPEEDKVLVAHIQRHGHGNWRALPKQAGLLRCGKSCRLRWINYLRPDIKRGNFSKEEEDTIIHLHELLGNRWSAIAARLPGRTDNEIKNVWHTHLKKRLDAPAQGGHVAASGGKKHKKPKSAKKPAAAAAAPPASPERSASSSVTESSMASSVAEEHGNAGISSASASVCAKEESSFTSASEEFQIDDSFWSETLSMPLDGYDVSMEPGDAFVAPPSADDMDYWLGVFMESGEAQDLPQI

SEQ ID NO: 9 - DNA, 1944 bp, Oryza sativa
atggctacttactattcaagccctggtagcgaaagagactcgcagaccatgtactcaagagacccaggcagtgcatcctatcctatgtcatcagctctagggaacttgctctatctgaacaatccatcttctggaccttatacggagttcagcggcattctgcagcctcagcagaattgcatggagatgcctggccctgggcatgcgtcagcaatgtcacaagacccctcttcaagggagtccgacatgctttcttcgcaccagggtcaacgctctttcagccatgtcaaagatatgaaaaatgagatgttgatgcatatgatggatggagcacaaggtagtggctctgagctcatccatgatgatgcccacactggttcacagcttgagtttggtgttctgaataatcacaactcatcaagtgttccatcaatgcaaagccaagggctatctctgagcttgaacacacaaatcatggcgccttccttaccgtactggtctataaagccagacatgttaacgccgcagtcttaccatgacaaccttagaggggaagacatgcggatgaagaatttgcagtccgaagcttcacgcgcaatccggaactcaaggtatctcaaggcagcacaagaactgcttgatgaggtcgtgagtgtttggaagagcataaaacaaaaggctcagaaagagaaggttgaatcaggaaaagcagatggcaaagagactgatggagggccaaaaagtgagggtgtatcttctaatccacaagaatctggcgctaatgcagcacctgagctttccactgctgagaagcaagagcttcagaacaaaatggcaaaactgatggcaatgttggatgaggtggaccggaaatacaagcattattaccaccaaatgcaaaccgtggtttcatcgtttgatgtggtagctggaccaggatctgcgaagccttacacagcagttgctcttcagacaatctcacggcactttcggtgcctgaaggatgctatcaatgatcagatcaatgttatcaggaaaaagcttggcgaggaagagaactcatctggcaaggagggaaaattaacccgcctccggtacattgatcagcagctaaggcaacaacgtgctttccaacagtacggtatgattccacaaaacgcctggagaccacagaggggactgcctgaaaactctgttacaattcttcgcgcttggctgtttgaacacttcctccatccgtacccaaaagattccgagaagttaatgcttgctaggcagactggcctgactaggagtcagatttcgaattggttcataaatgcccgtgtccgcctttggaaaccaatgattgaagacatgtacaaagaagagattggtgatttagagcaagactccaactcttcctctgacaatgcaccaagaagtaaggataaaatggcatcttcagaagacaaggaggatctcaaaaactccagggcacggatttgcgaaacaagccaattgagcgagtcgagaaccagcattggagcaatgaatgttggcggagcgcctgttggcttccagaatgagcccaatcctgatgacagtttcatgaacctgatgctgaaggaccagcgatcgaacgaagtggatggcggcctccttctccacaacactgtcgcgcagcactcagacgagaacgcccggttcatggcctatcacttggcagagcttgggagatacggcaatggcaatgtgtcgctgacactgggcttgcagcactccagcagcaacctcgttcctaatgctcagccaggtttccctggtgttaatgaggatgacatctacaatgctactgctcctcttggtgtcaccgtcgcatcttcggactatgattcgatgaatcagatggatcaacggcaacggtttgagcattcacctcttctgcatgattttgtggcctga

SEQ ID NO: 10 - protein, 647 aa, Oryza sativa
MATYYSSPGSERDSQTMYSRDPGSASYPMSSALGNLLYLNNPSSGPYTEFSGILQPQQNCMEMPGPGHASAMSQDPSSRESDMLSSHQGQRSFSHVKDMKNEMLMHMMDGAQGSGSELIHDDAHTGSQLEFGVLNNHNSSSVPSMQSQGLSLSLNTQIMAPSLPYWSIKPDMLTPQSYHDNLRGEDMRMKNLQSEASRAIRNSRYLKAAQELLDEVVSVWKSIKQKAQKEKVESGKADGKETDGGPKSEGVSSNPQESGANAAPELSTAEKQELQNKMAKLMAMLDEVDRKYKHYYHQMQTVVSSFDVVAGPGSAKPYTAVALQTISRHFRCLKDAINDQINVIRKKLGEEENSSGKEGKLTRLRYIDQQLRQQRAFQQYGMIPQNAWRPQRGLPENSVTILRAWLFEHFLHPYPKDSEKLMLARQTGLTRSQISNWFINARVRLWKPMIEDMYKEEIGDLEQDSNSSSDNAPRSKDKMASSEDKEDLKNSRARICETSQLSESRTSIGAMNVGGAPVGFQNEPNPDDSFMNLMLKDQRSNEVDGGLLLHNTVAQHSDENARFMAYHLAELGRYGNGNVSLTLGLQHSSSNLVPNAQPGFPGVNEDDIYNATAPLGVTVASSDYDSMNQMDQRQRFEHSPLLHDFVA

SEQ ID NO: 11 - DNA, 1005 bp, Oryza sativa
atgtgtggaggcgccatcctcgccgagttcatcccggcgccgtcgcgcgccgcggcggcgaccaagcgggtgaccgccagccacctgtggccggccggctccaagaacgccgcccgcggcaagagcaagagcaagaggcagcagaggagcttcgccgacgtcgacgacttcgaggccgccttcgagcagttcgacgatgactccgacttcgacgacgcggaggaagaagacgaaggacacttcgtgttcgcgtccaaatctcgtgtcgtcgccgggcacgacgggcgcgcggcggcgagggcggcgagcaagaagaagcgggggcggcacttccgaggcatccggcagcggccatgggggaagtgggcggcggagatccgcgacccgcacaagggcacgcgcgtctggctcggcacgttcaacaccccggaggaggccgcacgcgcctacgacgtcgaggcgcgccgcctccgcggcagcaaggccaaggtcaacttccccgccacgcccgccgccgcgcgcccacgccgcggcaacacgagagccaccgccgtgccaccgccggcgacagcacccgccgccgccccgccgcgcggactgaagcgagaattctcgccgcctgctgagaccgcgctacctttcttcaccaacggcttcgtcgacctgacgaccgccgcggcgccgccaccggccatgatgatgacgagctccttcaccgacagcgtcgccacgtcggagtccggcgggagccccgccaagaaggcgaggtccgacgacgtcgactcgtccgagggcagcgtcggcggcggcagcgacacgctgggtttcaccgacgagctggagttcgacccgttcatgctgttccagctcccctactccgacggctacgagtccatcgacagcctcttcgccgccggcgacgccaacagcgcgaacaccgacatgaacgccggcgtcaacctgtggagcttcgacgacttcccaatcgacggcgcccttttctga

SEQ ID NO: 12 - protein, 334 aa, Oryza sativa
MCGGAILAEFIPAPSRAAAATKRVTASHLWPAGSKNAARGKSKSKRQQRSFADVDDFEAAFEQFDDDSDFDDAEEEDEGHFVFASKSRVVAGHDGRAAARAASKKKRGRHFRGIRQRPWGKWAAEIRDPHKGTRVWLGTFNTPEEAARAYDVEARRLRGSKAKVNFPATPAAARPRRGNTRATAVPPPATAPAAAPPRGLKREFSPPAETALPFFTNGFVDLTTAAAPPPAMMMTSSFTDSVATSESGGSPAKKARSDDVDSSEGSVGGGSDTLGFTDELEFDPFMLFQLPYSDGYESIDSLFAAGDANSANTDMNAGVNLWSFDDFPIDGALF

SEQ ID NO: 13 - DNA, 804 bp, Oryza sativa
atgggctctgctccgttcggtgacgccgtcgccggcggtgggctctatgagtaccaggggtatcacggtggcttcgccggcggccatggtctcggccaaccggccggccgagcgcccgcgttggacgacggtgaaactgaggggatggacgcatccgccgccgccgcagtcgccgccatggagatggcaaagaggaattgtggaggcggccgggaggagaaggcggcgatggcgctcaagagccacagcgaggcggagcggcggcggcgggagaggatcaacgcccacctcgccacgctccgcaccatggtgccctgcaccgacaagatggacaaggccgcgctgctggctgaggtggtgggccacgtcaagaagctgaagagcgccgcggcgcgcgtcggccggcgcgccaccgtcccgtccggcgccgacgaggtcgccgtcgacgaagcgtccgccaccggaggcggaggagaaggccccctcctcctcagggcgacgctgagctgcgacgaccgcgccgacctcttcgtggacgtcaagcgcgcgctgcagccgctcgggctggaggtggtcggctccgaggtcaccacgctgggcggccgcgtccgcctcgccttcctggtctcgtgcgggtctcgcggcggcgccgccgccgccgccatggcctcggtgcgccacgcgctccagtccgtgctcgacaaggccagctccggcttcgacttcgcgcccagggcggcgtcgctgctgggcagcaaacgccggaaggtctccacgttcgagtcatcgagctcctcgtcttga

SEQ ID NO: 14 - protein, 267 aa, Oryza sativa
MGSAPFGDAVAGGGLYEYQGYHGGFAGGHGLGQPAGRAPALDDGETEGMDASAAAAVAAMEMAKRNCGGGREEKAAMALKSHSEAERRRRERINAHLATLRTMVPCTDKMDKAALLAEVVGHVKKLKSAAARVGRRATVPSGADEVAVDEASATGGGGEGPLLLRATLSCDDRADLFVDVKRALQPLGLEVVGSEVTTLGGRVRLAFLVSCGSRGGAAAAAMASVRHALQSVLDKASSGFDFAPRAASLLGSKRRKVSTFESSSSSS

SEQ ID NO: 15 - DNA, 1287 bp, Oryza sativa
atggggagacattcctgctgctacaagcagaagctgaggaaggggctctggtcacctgaggaggatgagaagctcatgaaccacataaccaagcatggccatggctgctggagcaccgttccaaaacttgcagggcttcagagatgtggcaaaagctgcaggctgagatggataaactacttgaggcctgacctaaagagaggtgcattctctcaggaggaagaggacctcatcgttgagctccatgctgttctgggaaacaggtggtctcaaattgcaacacggctgcctggaagaactgacaatgagatcaagaatctctggaattcatgcatcaagaagaagctgcggcagaaaggcattgacccaaacacccacaagccccttgccgaggttgatcgaagcaaagcaactcccaccatcagcaatgacaggacctctgaatccagcgatgttgacccatctagtggcgttgctcttcacaacttgagccatcttctgagcgagacggcgcaatcatcagagctgctgccagtgaaggtaactaaacctcgcacacaagctccgggcttggcacgtctgaaggtgccaccgaaggaactatttctagaccagcttacttctggccatgagaacctccctagctgccgttcatctggcccaattcccaatttcccgttccagcagctgttgtgctacaataatgattttaacagcatggacgttggaaacaggaattcactctggtataaccagaatgagtccagtagcagcacaatttccactgtgatgcccccagtttcaccatcaactctctcgacatcaacaggccttaatccatcaccagacaatgcaaactctcgaggcactggcatccacaattcccaattctattgggacaccaacaatcctagcagcagtagcagtacagggagtagtggtaacaacgggttgggattcgagctgcaaagcacaagctcccttttggagaccaacatattcccatggtcagatttagcaccagaaaaggatagccaagcacaactagaggaagaactcaagtggcctgacttgctccatggaacgttctcagagatgccagcgcccatgcagaaccttagccagtcactgtatgaagacgttgtcaaagcagagagccaattcaacatggaggggctctgtgcagcttggtctcaaaacctgctgccacagcaacatcttccagtagtatcagatatgtatgacaaggatttgcaaaggatgtccttgtcttttgagaatatctag

SEQ ID NO: 16 - protein, 428 aa, Oryza sativa
MGRHSCCYKQKLRKGLWSPEEDEKLMNHITKHGHGCWSTVPKLAGLQRCGKSCRLRWINYLRPDLKRGAFSQEEEDLIVELHAVLGNRWSQIATRLPGRTDNEIKNLWNSCIKKKLRQKGIDPNTHKPLAEVDRSKATPTISNDRTSESSDVDPSSGVALHNLSHLLSETAQSSELLPVKVTKPRTQAPGLARLKVPPKELFLDQLTSGHENLPSCRSSGPIPNFPFQQLLCYNNDFNSMDVGNRNSLWYNQNESSSSTISTVMPPVSPSTLSTSTGLNPSPDNANSRGTGIHNSQFYWDTNNPSSSSSTGSSGNNGLGFELQSTSSLLETNIFPWSDLAPEKDSQAQLEEELKWPDLLHGTFSEMPAPMQNLSQSLYEDVVKAESQFNMEGLCAAWSQNLLPQQHLPVVSDMYDKDLQRMSLSFENI

SEQ ID NO: 17 - DNA, 1284 bp, Oryza sativa
atggggaggcattcttgctgctacaagcagaaattgaggaaggggctctggtcccctgaggaggatgagaagctcatgaatcacataaccaagcatggccatggctgctggagttctgttcctaaacttgcagggctccaaagatgcggcaagagctgcaggctgaggtggataaactatctgaggcctgacctcaagagaggtgcattctctcaggaggaagaagacctcatcattgagctccatgctgtccttgggaacaggtggtctcagattgcagctcagctgcctgggaggactgacaacgagatcaagaatctctggaattcttgcatcaagaagaagctccggcagaaaggcattgatcccaacacccacaagcccctcaccgaggctgatcgcaggggagcagcacccacagtcagcaccgagaggacctcagggtccagcgacgtcaacccgtcaagtgctggtgctctagggaacttgagccacctcctcagcgagacagcacaatcatcaatgctgctgccggtgtatgataagaatcaccctgaaactgcaagcttgccacgccctaaggtgccaccaaaggagttgtttcttgaccagcttactgccggtcacgagagcccatcaagttgccgctcatcaggtccaaccctgtattttcctttccagcaaccattaggctacagtaatgaatgtggcactggggatggtgcgagtatgaattcactctggttcaaccagaatgatttcaactgcagcacaatctccaccgtgatgccaccagtttcaccatcagccctctcaacatcaatggggctgaatcttcccccagaaaatcctcgtcacggaggcactggcattggcaacaccccgttctactgggatggttccaatcctagcagcagcggcagtactgggagcagcggaagcaacagtatgggattcgagccacagagcacaacctcaattttggagaacagtgtattcccatggacagatatcggacaagagaaagataccagagttcacttagtggaggaactcaagtggcctgatttgctccatggaacctttgctgaggcgacgacagccatgcagaaccagagccaatcattatacgatgatgtgatcaaggcagagagccaattcaacatggaagggatatgtgcctcttggttccagaatcagcaaccacaacaacagctgcaagcagcatcagacatgtatgataaagacttgcaaagattgcccttgtcttttgaacatatctag

SEQ ID NO: 18 - protein, 427 aa, Oryza sativa
MGRHSCCYKQKLRKGLWSPEEDEKLMNHITKHGHGCWSSVPKLAGLQRCGKSCRLRWINYLRPDLKRGAFSQEEEDLIIELHAVLGNRWSQIAAQLPGRTDNEIKNLWNSCIKKKLRQKGIDPNTHKPLTEADRRGAAPTVSTERTSGSSDVNPSSAGALGNLSHLLSETAQSSMLLPVYDKNHPETASLPRPKVPPKELFLDQLTAGHESPSSCRSSGPTLYFPFQQPLGYSNECGTGDGASMNSLWFNQNDFNCSTISTVMPPVSPSALSTSMGLNLPPENPRHGGTGIGNTPFYWDGSNPSSSGSTGSSGSNSMGFEPQSTTSILENSVFPWTDIGQEKDTRVHLVEELKWPDLLHGTFAEATTAMQNQSQSLYDDVIKAESQFNMEGICASWFQNQQPQQQLQAASDMYDKDLQRLPLSFEHI